(19) United States
(12) Patent Application Publication
(10) Pub. No.: US 2023/0372536 A1
(43) Pub. Date: Nov. 23, 2023

(54) NANOMATERIAL AND METHODS OF USE THEREOF

(71) Applicant: UNIVERSITY OF CONNECTICUT, Farmington, CT (US)

(72) Inventor: Yupeng CHEN, Sharon, MA (US)

(21) Appl. No.: 18/248,021

(22) PCT Filed: Oct. 13, 2021
(86) PCT No.: PCT/US2021/054800

§ 371 Date: Apr. 5, 2023

Related U.S. Application Data
(60) Provisional application No. 63/090,832, filed on Oct. 13, 2020.

(51) Int. Cl. A61K 47/69; B82B 1/00; B82Y 5/00

(52) U.S. Cl. CPC A61K 47/6957; B82B 1/001; B82Y 5/00

(57) ABSTRACT

Disclosed herein are self-assembled nanomaterials that include a Janus base nanotube having a biologically active molecule noncovalently adhered thereto, wherein the biologically active molecule is an extracellular matrix (ECM) molecule, a bioactive molecule, or a combination thereof.

### CROSS-REFERENCE TO RELATED APPLICATIONS

This application is a National Stage Entry of International Patent Application No. PCT/US2021/054800, filed on 13 Oct. 2021 and published as International Patent Application Publication No. WO 2022/081721 A1, which claims the priority to, and the benefit of, U.S. Provisional Application No. 63/090,832, filed on Oct. 13, 2020, the entire contents of each is incorporated herein by reference.

### STATEMENT OF GOVERNMENT SUPPORT

This invention was made with government support under 7R01AR072024 and AR069383 awarded by the National Institutes of Health and CBET-1905785 and CMMI-2025362 awarded by the National Science Foundation. The government has certain rights in the invention.

### BACKGROUND

Biomaterials such as hydrogels have been used as three-dimensional matrices for stem cells due to their biocompatibility and ability to mimic the extracellular matrix (ECM). While hydrogels can support cell growth, hydrogels are homogenous jelly-like materials and not solid scaffolds. For certain applications solid scaffolds, and particularly injectable solid scaffolds, are in great need.

### SUMMARY

In an aspect, disclosed herein are self-assembled nanomaterials comprising a Janus base nanotube (JBNT) having a biologically active molecule noncovalently adhered thereto, wherein the biologically active molecule comprises an extracellular matrix (ECM) molecule, a bioactive molecule, or a combination thereof.

Also disclosed herein are injectable compositions comprising the self-assembled nanomaterials described above and a pharmaceutically acceptable carrier.

Also disclosed are tissue chips comprising a microfluidic cell and the self-assembled nanomaterials described above.

In another aspect, disclosed herein are methods of tissue engineering comprising injecting into a tissue the injectable composition described above.

The above-described and other features will be appreciated and understood by those skilled in the art from the following detailed description, drawings, and appended claims.

### BRIEF DESCRIPTION OF THE FIGURES

FIG. 1 shows formulation development and the camera images of the JBNT/matrilin3(Matn3) nanomaterial matrices (NM).

FIG. 2 shows characterization of the JBNT/Matn3 NM.

FIG. 3 shows transmission electron microscopy (TEM) images of the NM.

FIG. 4 shows Widefield images of the NM.

FIG. 5 are graphs showing cell adhesion and density on the NM.

FIGS. 6A and 6B show fluorescence microscopy images of the double-layered NM from JBNTs, Matn3, and TGFβ.

FIGS. 7A, 7B, and 7C show zeta potential (FIG. 7A), UV-Vis (FIG. 7B) and TEM analysis (FIG. 7C) of the double-layered NM from JBNTs, Matn3 and TGFβ.

FIGS. 8A, 8B, 8C, and 8D show cell adhesion images (FIGS. 8A-8B) and cell adhesion numbers (per mm²) (FIGS. 8C-8D) of the double-layered NM from JBNTs, Matn3 and TGFβ.

FIG. 9 shows cell morphology analysis of the double-layered NM from JBNTs, Matn3 and TGFβ.

FIGS. 10A, 10B, and 10C shows the chemical structure of JBNTs (FIG. 10A); the formation process of J/T/M NM by self-assembly (FIG. 10B); and the biological activity of J/T/M NM co-cultured with mesenchymal stem cells (FIG. 10C).

FIGS. 11A, 11B, and 11C shows the development and characterization of J/T/M NM. FIG. 11A shows the zeta-potential of matrilin-3, matrilin-3/TGF-β1 mixture, and J/T/M NM. FIG. 11B shows ultraviolet-visible (UV-Vis) absorption spectra of matrilin-3, TGF-β1, JBNTs, matrilin3/JBNT complex, TGF-β1/JBNT complex, and J/T/M NM. FIG. 11C shows transmission electron microscopy (TEM) images of JBNTs and J/T/M NM at two different magnifications.

FIGS. 12A, 12B, 12C, and 12D shows hMSC adhesion behavior test and analysis. FIG. 12A shows optical microscope images of hMSCs cultured on the surface of pre-coated agarose gel. FIG. 12B shows confocal images of hMSCs cultured on chambered coverglass coated with different materials. FIG. 12C shows statistical analysis of cell adhesion numbers. FIG. 12D shows statistical analysis of cell morphology. N≥3. *P<0.05, **P<0.01, ***P<0.001, ****P<0.0001 compared with negative controls (NC).

FIG. 13 shows statistical analysis map of cell shape parameters among different groups of hMSCs. N≥3. *P<0.05, **P<0.01, ****P<0.0001.

FIG. 14 shows statistical analysis of cell proliferation. Cell number statistics of hMSCs after being incubated with different materials for 1 day, 3 days, or 5 days. *P<0.05, **P<0.01, ***P<0.001, ****P<0.0001. N=6.

FIGS. 15A and 15B show Alcian blue staining of cartilage tissue constructs and quantified analysis of stained hMSCs after 15 days of differentiation. FIG. 15A shows light microscopy images of cartilage tissue constructs containing Alcian blue stained hMSCs. FIG. 15B shows total numbers and anchored percentage analysis for the hMSCs in cartilage tissue constructs. Scale bars: 50 μm.

FIGS. 16A, 16B, 16C, 16D, and 16E show promotion of chondrogenesis and prevention of hypertrophy by J/T/M NM in a 3D culture system evaluated by real-time PCR and immunostaining. Real-time PCR was performed on samples harvested at 15 days to evaluate the gene expression of a chondrogenic marker (aggrecan (FIG. 16A); COL2A1 (FIG. 16B)) and a hypertrophic marker (COL10A1 (FIG. 16C); IHH (FIG. 16D)). Expression of the gene of interest was normalized by expression of the housekeeping gene GAPDH. *P<0.05, ***P<0.001, ****P<0.0001. N=3. FIG. 16E shows confocal images of Type X collagen immunostaining.

FIGS. 17A, 17B, and 17C show J/T/M NM stability test. FIG. 17A shows ultraviolet-visible (UV-Vis) absorption spectra of J/T/M NM tested in 15 days. FIG. 17B shows UV-Vis absorption spectra of the J/T/M NM and different control groups (including JBNTs, matrilin-3, TGF-β1 and JBNT/matrilin-3 complex) tested on day 0, day 9, and day 15. FIG. 17C shows the percentage of TGF-β1 remained in the J/T/M NM after 15 days (determined by an enzyme-linked immunoassay (ELISA) kit).

FIG. 18 shows a demonstration of solid and flexible J/T/M NM fibers in water.

FIGS. 19A and 19B shows an in vitro cytotoxicity assay using JBNT solution. Relative viabilities of hMSCs (FIG. 19A) and human chondrocytes (C28/I2 cell line; (FIG. 19B)) following incubation with different concentrations of JBNT solution.

FIGS. 20A, 20B, and 20C show human chondrocyte adhesion behavior test and analysis. FIG. 20A shows confocal images of human chondrocytes cultured on a chambered coverglass precoated with different materials. Scale bars: 50 μm. FIG. 20B shows statistical analysis of cell adhesion numbers. FIG. 20C shows statistical analysis of cell morphology. N≥3. *P<0.05, ** P<0.01, ***P<0.001.

FIG. 21 shows statistical analysis map of cell shape parameters among different groups of chondrocytes. N≥3. *P<0.05, **P<0.01, ***P<0.001.

FIG. 22 shows the standard curve of absorption intensity for hMSCs tested with the CCK-8 assay.

FIG. 23 shows Alcian blue staining of cartilage tissue constructs. Scale bars: 50 μm.

FIGS. 24A, 24B, and 24C show fluorescence spectra and confocal images of J/T/M NM formed with JBNTs and fluorescently labeled proteins. FIG. 24A shows 3D confocal images of JBNTs/TGF-β1-Alex Fluor 488/matrilin-3-Alex Fluor 555 NM. FIG. 24B shows 2D confocal images of JBNTs/TGF-β1-Alex Fluor 488/matrilin-3-Alex Fluor 555 NM in different channels. FIG. 24C shows FRET process between fluorescent dye-labeled proteins was characterized by the fluorescence spectra.

### DETAILED DESCRIPTION

Osteoporosis is a common and frequently occurring disease, and the fractures that occur in patients with osteoporosis not only cause great pain and are slow to recover from, but also bring a heavy economic burden to the patients. Activation and migration of mesenchymal stem cells (MSCs) have been shown to play an important role in fracture healing, however, it has been a challenge to promote and guide endogenous MSCs to the fracture site and to promote adhesion and function at the target location. As attracting MSCs to migrate into and adhere within the fracture site is the first step in bone regeneration, a successful tissue engineering scaffold should be able to enhance stem cell anchorage, which includes supporting migration and adhesion. This is important for cell differentiation and function. Without a biomaterial or biochemical cues to guide them, only a small portion of injected MSCs reaches the target tissue and remains at the desired location, especially in the case of systemic administration. Although variously engineered scaffolds have been used to facilitate MSCs migration and adhesion, some fracture locations (such as a growth plate fracture in the middle of a long bone) are not easy to access and do not readily accommodate conventional grafting materials or scaffolds which are prefabricated. Moreover, prefabricated scaffolds may not fit perfectly into an irregularly shaped fracture. Therefore, what is needed is a nanomaterial that is not only biomimetic but can self-assemble in situ and thereby be injectable directly into the target area.

Described herein is a family of injectable nanomaterial matrices (NMs) that can create a microenvironment tailored for stem cell differentiation or tissue cell functions. These nanomaterials can be used for “difficult-to-reach” locations such as deep tissue injuries (as a tissue repair therapy) or the microchannels of tissue chips (for disease modeling and drug screen). Unlike prior art injectable materials such as injectable hydrogels—which are homogenous jelly-like materials—the NMs described herein are porous solid meshes. The NMs described herein are particularly useful for tissue regeneration of deep tissue injuries (such as growth plate fracture repair and brain regeneration after stroke) and can also be used for tissue chips for drug screening.

### Definitions

Throughout the present specification and the accompanying claims the words “comprise,” “include,” and “have” and variations thereof such as “comprises,” “comprising,” “includes,” “including,” “has,” and “having” are to be interpreted inclusively. That is, these words are intended to convey the possible inclusion of other elements or integers not specifically recited, where the context allows. No language in the specification should be construed as indicating any non-claimed element essential to the practice of the invention.

The terms “a,” “an,” and “the” and similar referents used in the context of describing the invention (especially in the context of the following claims) are to be construed to cover both the singular and the plural, unless otherwise indicated herein or clearly contradicted by context.

The terms first, second, etc. as used herein are not meant to denote any particular ordering, but simply for convenience to denote a plurality of, for example, layers.

Recitation of ranges of values herein are merely intended to serve as a shorthand method of referring individually to each separate value falling within the range, unless otherwise indicated herein, each individual value is incorporated into the specification as if it were individually recited herein. Ranges may be expressed herein as from “about” (or “approximately”) one particular value, and/or to “about” (or “approximately”) another particular value. When such a range is expressed, another embodiment includes from the one particular value and/or to the other particular value. Similarly, when values are expressed as approximations, by use of the antecedent “about” or “approximately” it will be understood that the particular value forms another embodiment. It will be further understood that the endpoints of each of the ranges are disclosed both in relation to the other endpoint, and independently of the other endpoint.

All methods described herein can be performed in any suitable order unless otherwise indicated herein or otherwise clearly contradicted by context. Further, all methods described herein and having more than one step can be performed by more than one person or entity. Thus, a person or an entity can perform step (a) of a method, another person or another entity can perform step (b) of the method, and a yet another person or a yet another entity can perform step (c) of the method, etc. The use of any and all examples, or exemplary language (e.g., “such as”) provided herein is intended merely to better illuminate the invention and does not pose a limitation on the scope of the invention otherwise claimed.

Units, prefixes, and symbols are denoted in their Systeme International de Unites (SI) accepted form.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

The headings used herein are for organizational purposes only and are not meant to be used to limit the scope of the description or the claims, which can be had by reference to the specification as a whole. Accordingly, the terms defined immediately below are more fully defined by reference to the specification in its entirety.

Illustrations are for the purpose of describing a preferred embodiment of the invention and are not intended to limit the invention thereto.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or 5% of the stated value.

As used herein, the term “administering” means the actual physical introduction of a composition into or onto (as appropriate) a host or cell. Any and all methods of introducing the composition into the host or cell are contemplated according to the invention; the method is not dependent on any particular means of introduction and is not to be so construed. Means of introduction are well-known to those skilled in the art, and also are exemplified herein.

As used herein, “optional” or “optionally” means that the subsequently described event or circumstance may or may not occur, and that the description includes instances where said event or circumstance occurs and instances where it does not.

As used herein, the term “pharmaceutically acceptable” refers to compositions that are physiologically tolerable and do not typically produce an allergic or similar untoward reaction when administered to a subject, preferably a human subject. Preferably, as used herein, the term “pharmaceutically acceptable” means approved by a regulatory agency of a federal or state government or listed in the U.S. Pharmacopeia or other generally recognized pharmacopeia for use in animals, and more particularly in humans.

As used herein, the terms “treat,” “treating,” and “treatment” include inhibiting the pathological condition, disorder, or disease, e.g., arresting or reducing the development of the pathological condition, disorder, or disease or its clinical symptoms; or relieving the pathological condition, disorder, or disease, e.g., causing regression of the pathological condition, disorder, or disease or its clinical symptoms. These terms also encompass therapy and cure. Treatment means any way the symptoms of a pathological condition, disorder, or disease are ameliorated or otherwise beneficially altered. Preferably, the subject in need of such treatment is a mammal, preferably a human.

### Chemical Definitions

The term “amino acid” refers to a molecule containing both an amino group and a carboxyl group. Exemplary amino acids include, without limitation, both the D- and L-isomers of the naturally-occurring amino acids, as well as non-naturally occurring amino acids prepared by organic synthesis or other metabolic routes. The term amino acid, as used herein, includes without limitation, α-amino acids, natural amino acids, non-natural amino acids, and amino acid analogs.

The term “α-amino acid” refers to a molecule containing both an amino group and a carboxyl group bound to a carbon which is designated the α-carbon.

The term “β-amino acid” refers to a molecule containing both an amino group and a carboxyl group in a β configuration.

The term “naturally occurring amino acid” refers to any one of the twenty amino acids commonly found in peptides synthesized in nature, and known by the one letter abbreviations A, R, N, C, D, Q, E, G, H, I, L, K, M, F, P, S, T, W, Y and V.

The following table shows a summary of the properties of natural amino acids:

| | 3- | 1- | | Side-chain | |
|---|---|---|---|---|---|
| | Letter | Letter | Side-chain | charge | Hydropathy |
| Amino Acid | Code | Code | Polarity | (pH 7.4) | Index |
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | polar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive (10%) | −3.2 |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

“Hydrophobic amino acids” include small hydrophobic amino acids and large hydrophobic amino acids. “Small hydrophobic amino acid” are glycine, alanine, proline, and analogs thereof “Large hydrophobic amino acids” are valine, leucine, isoleucine, phenylalanine, methionine, tryptophan, and analogs thereof. “Polar amino acids” are serine, threonine, asparagine, glutamine, cysteine, tyrosine, and analogs thereof. “Charged amino acids” are lysine, arginine, histidine, aspartate, glutamate, and analogs thereof.

The term “amino acid analog” refers to a molecule which is structurally similar to an amino acid and that can be substituted for an amino acid in the formation of a peptidomimetic macrocycle. Amino acid analogs include, without limitation, 3-amino acids, and amino acids where the amino or carboxy group is substituted by a similarly reactive group (e.g., substitution of the primary amine with a secondary or tertiary amine, or substitution of the carboxy group with an ester).

The term “non-natural amino acid” refers to an amino acid that is not one of the twenty amino acids commonly found in peptides synthesized in nature, and known by the one letter abbreviations A, R, N, C, D, Q, E, G, H, I, L, K, M, F, P, S, T, W, Y and V. Non-natural amino acids or amino acid analogs include, without limitation, structures according to the following:

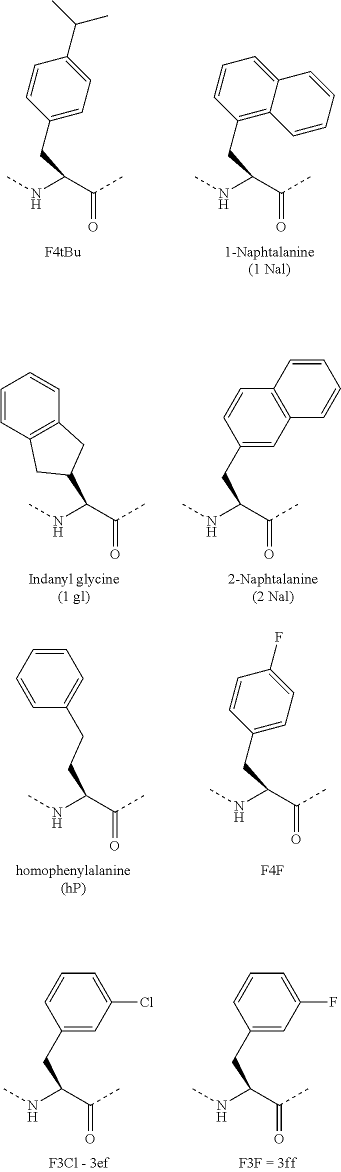

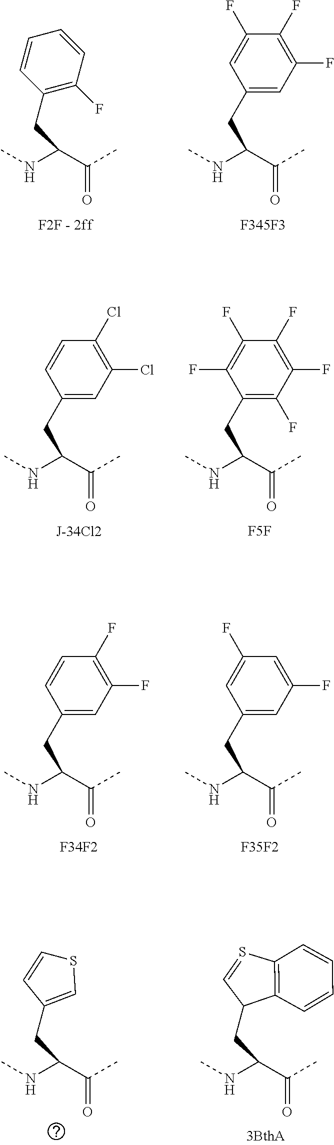

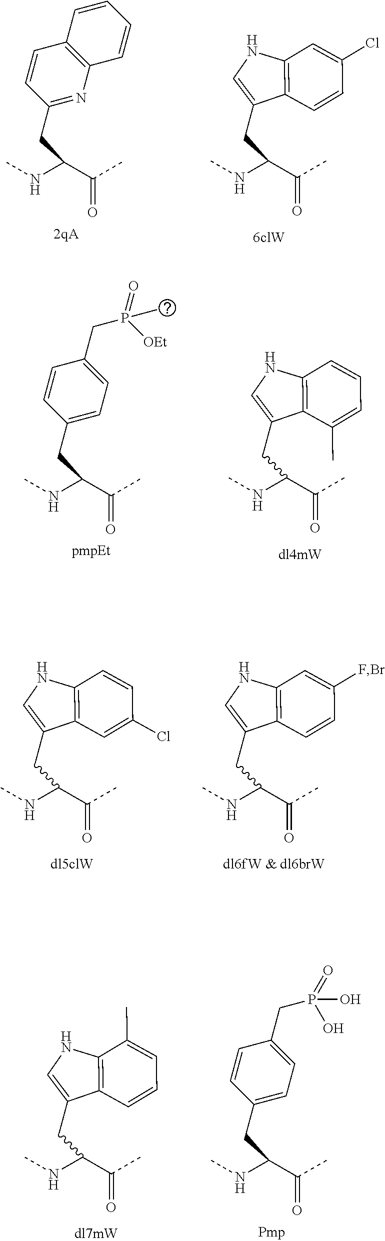

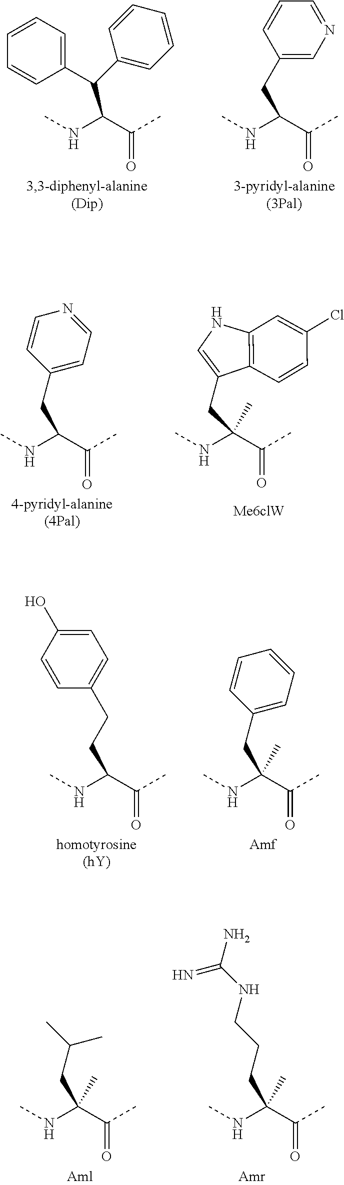

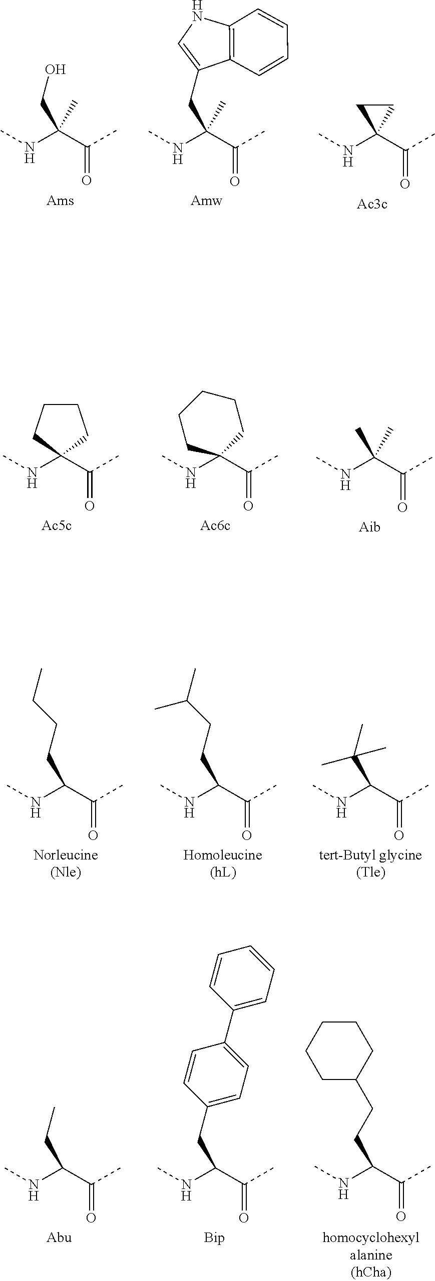

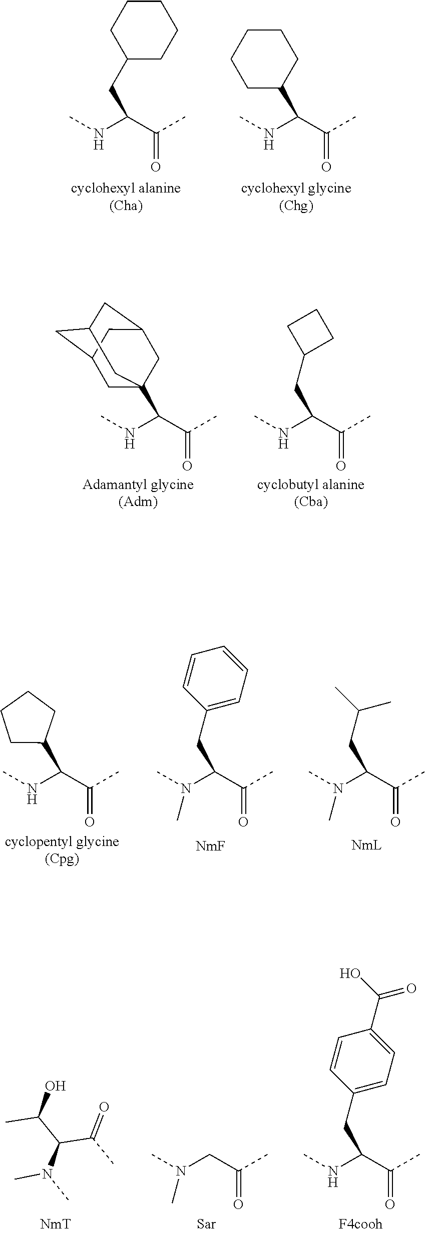

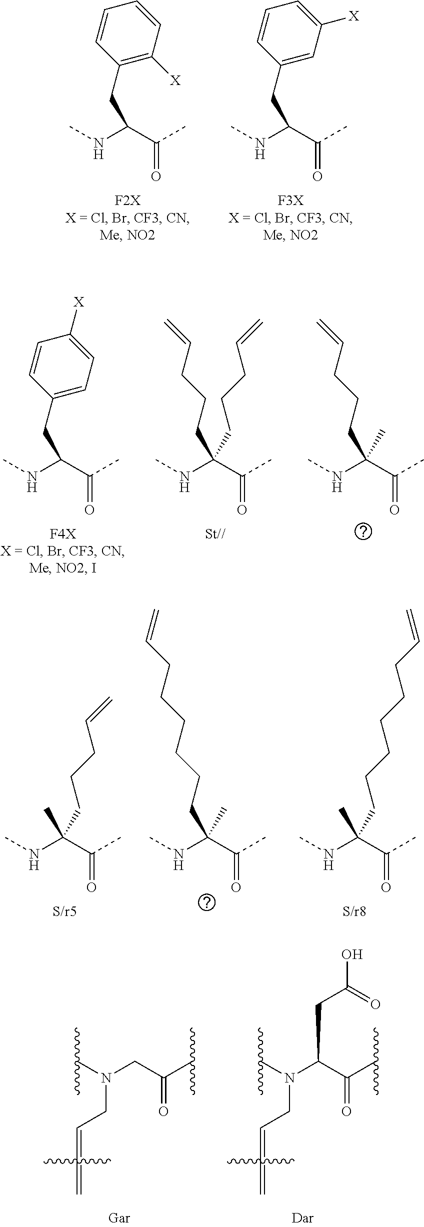

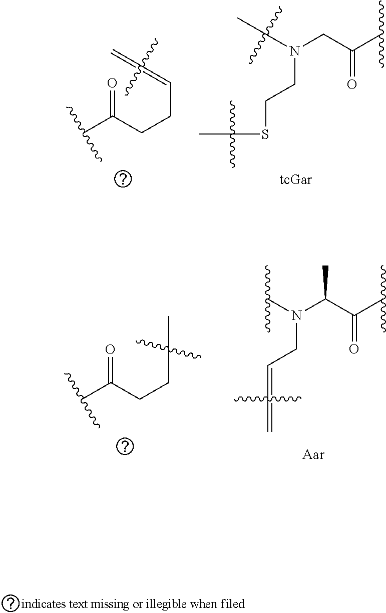

Amino acid analogs include β-amino acid analogs. Examples of β-amino acid analogs include, but are not limited to, the following: cyclic β-amino acid analogs; β-alanine; (R)-β-phenylalanine; (R)-1,2,3,4-tetrahydro-isoquinoline-3-acetic acid; (R)-3-amino-4-(1-naphthyl)-butyric acid; (R)-3-amino-4-(2,4-dichlorophenyl)butyric acid; (R)-3-amino-4-(2-chlorophenyl)-butyric acid; (R)-3-amino-4-(2-cyanophenyl)-butyric acid; (R)-3-amino-4-(2-fluorophenyl)-butyric acid; (R)-3-amino-4-(2-furyl)-butyric acid; (R)-3-amino-4-(2-methylphenyl)-butyric acid; (R)-3-amino-4-(2-naphthyl)-butyric acid; (R)-3-amino-4-(2-thienyl)-butyric acid; (R)-3-amino-4-(2-trifluoromethylphenyl)-butyric acid; (R)-3-amino-4-(3,4-dichlorophenyl)butyric acid; (R)-3-amino-4-(3,4-difluorophenyl)butyric acid; (R)-3-amino-4-(3-benzothienyl)-butyric acid; (R)-3-amino-4-(3-chlorophenyl)-butyric acid; (R)-3-amino-4-(3-cyanophenyl)-butyric acid; (R)-3-amino-4-(3-fluorophenyl)-butyric acid; (R)-3-amino-4-(3-methylphenyl)-butyric acid; (R)-3-amino-4-(3-pyridyl)-butyric acid; (R)-3-amino-4-(3-thienyl)-butyric acid; (R)-3-amino-4-(3-trifluoromethylphenyl)-butyric acid; (R)-3-amino-4-(4-bromophenyl)-butyric acid; (R)-3-amino-4-(4-chlorophenyl)-butyric acid; (R)-3-amino-4-(4-cyanophenyl)-butyric acid; (R)-3-amino-4-(4-fluorophenyl)-butyric acid; (R)-3-amino-4-(4-iodophenyl)-butyric acid; (R)-3-amino-4-(4-methylphenyl)-butyric acid; (R)-3-amino-4-(4-nitrophenyl)-butyric acid; (R)-3-amino-4-(4-pyridyl)-butyric acid; (R)-3-amino-4-(4-trifluoromethylphenyl)-butyric acid; (R)-3-amino-4-pentafluoro-phenylbutyric acid; (R)-3-amino-5-hexenoic acid; (R)-3-amino-5-hexynoic acid; (R)-3-amino-5-phenylpentanoic acid; (R)-3-amino-6-phenyl-5-hexenoic acid; (S)-1,2,3,4-tetrahydro-isoquinoline-3-acetic acid; (S)-3-amino-4-(1-naphthyl)-butyric acid; (S)-3-amino-4-(2,4-dichlorophenyl)butyric acid; (S)-3-amino-4-(2-chlorophenyl)-butyric acid; (S)-3-amino-4-(2-cyanophenyl)-butyric acid; (S)-3-amino-4-(2-fluorophenyl)-butyric acid; (S)-3-amino-4-(2-furyl)-butyric acid; (S)-3-amino-4-(2-methylphenyl)-butyric acid; (S)-3-amino-4-(2-naphthyl)-butyric acid; (S)-3-amino-4-(2-thienyl)-butyric acid; (S)-3-amino-4-(2-trifluoromethylphenyl)-butyric acid; (S)-3-amino-4-(3,4-dichlorophenyl)butyric acid; (S)-3-amino-4-(3,4-difluorophenyl)butyric acid; (S)-3-amino-4-(3-benzothienyl)-butyric acid; (S)-3-amino-4-(3-chlorophenyl)-butyric acid; (S)-3-amino-4-(3-cyanophenyl)-butyric acid; (S)-3-amino-4-(3-fluorophenyl)-butyric acid; (S)-3-amino-4-(3-methylphenyl)-butyric acid; (S)-3-amino-4-(3-pyridyl)-butyric acid; (S)-3-amino-4-(3-thienyl)-butyric acid; (S)-3-amino-4-(3-trifluoromethylphenyl)-butyric acid; (S)-3-amino-4-(4-bromophenyl)-butyric acid; (S)-3-amino-4-(4-chlorophenyl)-butyric acid; (S)-3-amino-4-(4-cyanophenyl)-butyric acid; (S)-3-amino-4-(4-fluorophenyl)-butyric acid; (S)-3-amino-4-(4-iodophenyl)-butyric acid; (S)-3-amino-4-(4-methylphenyl)-butyric acid; (S)-3-amino-4-(4-nitrophenyl)-butyric acid; (S)-3-amino-4-(4-pyridyl)-butyric acid; (S)-3-amino-4-(4-trifluoromethylphenyl)-butyric acid; (S)-3-amino-4-pentafluoro-phenylbutyric acid; (S)-3-amino-5-hexenoic acid; (S)-3-amino-5-hexynoic acid; (S)-3-amino-5-phenylpentanoic acid; (S)-3-amino-6-phenyl-5-hexenoic acid; 1,2,5,6-tetrahydropyridine-3-carboxylic acid; 1,2,5,6-tetrahydropyridine-4-carboxylic acid; 3-amino-3-(2-chlorophenyl)-propionic acid; 3-amino-3-(2-thienyl)-propionic acid; 3-amino-3-(3-bromophenyl)-propionic acid; 3-amino-3-(4-chlorophenyl)-propionic acid; 3-amino-3-(4-methoxyphenyl)-propionic acid; 3-amino-4,4,4-trifluoro-butyric acid; 3-aminoadipic acid; D-β-phenylalanine; β-leucine; L-β-homoalanine; L-β-homoaspartic acid γ-benzyl ester; L-β-homoglutamic acid δ-benzyl ester; L-β-homoisoleucine; L-β-homoleucine; L-β-homomethionine; L-β-homophenylalanine; L-β-homoproline; L-β-homotryptophan; L-β-homovaline; L-Nω-benzyloxycarbonyl-β-homolysine; Nω-L-β-homoarginine; O-benzyl-L-β-homohydroxyproline; O-benzyl-L-β-homoserine; O-benzyl-L-β-homothreonine; O-benzyl-L-β-homotyrosine; γ-trityl-L-β-homoasparagine; (R)-β-phenylalanine; L-β-homoaspartic acid γ-t-butyl ester; L-β-homoglutamic acid δ-t-butyl ester; L-Nω-β-homolysine; Nδ-trityl-L-β-homoglutamine; Nω-2,2,4,6,7-pentamethyl-dihydrobenzofuran-5-sulfonyl-L-β-homoarginine; O-t-butyl-L-β-homohydroxy-proline; O-t-butyl-L-β-homoserine; O-t-butyl-L-β-homothreonine; O-t-butyl-L-β-homotyrosine; 2-aminocyclopentane carboxylic acid; and 2-aminocyclohexane carboxylic acid.

Amino acid analogs include analogs of alanine, valine, glycine or leucine. Examples of amino acid analogs of alanine, valine, glycine, and leucine include, but are not limited to, the following: α-methoxyglycine; α-allyl-L-alanine; α-aminoisobutyric acid; α-methyl-leucine; β-(1-naphthyl)-D-alanine; β-(1-naphthyl)-L-alanine; β-(2-naphthyl)-D-alanine; β-(2-naphthyl)-L-alanine; 1-(2-pyridyl)-D-alanine; β-(2-pyridyl)-L-alanine; β-(2-thienyl)-D-alanine; β-(2-thienyl)-L-alanine; β-(3-benzothienyl)-D-alanine; β-(3-benzothienyl)-L-alanine; β-(3-pyridyl)-D-alanine; β-(3-pyridyl)-L-alanine; β-(4-pyridyl)-D-alanine; β-(4-pyridyl)-L-alanine; 1-chloro-L-alanine; 1-cyano-L-alanin; 3-cyclohexyl-D-alanine; 3-cyclohexyl-L-alanine; 3-cyclopenten-1-yl-alanine; 3-cyclopentyl-alanine; 3-cyclopropyl-L-Ala-OH.dicyclohexylammonium salt; β-t-butyl-D-alanine; β-t-butyl-L-alanine; γ-aminobutyric acid; L-α,β-diaminopropionic acid; 2,4-dinitro-phenylglycine; 2,5-dihydro-D-phenylglycine; 2-amino-4,4,4-trifluorobutyric acid; 2-fluoro-phenylglycine; 3-amino-4,4,4-trifluoro-butyric acid; 3-fluoro-valine; 4,4,4-trifluoro-valine; 4,5-dehydro -L-leu-OH.dicyclohexylammonium salt; 4-fluoro-D-phenylglycine; 4-fluoro-L-phenylglycine; 4-hydroxy-D-phenylglycine; 5,5,5-trifluoro-leucine; 6-aminohexanoic acid; cyclopentyl-D-Gly-OH.dicyclohexylammonium salt; cyclopentyl-Gly-OH.dicyclohexylammonium salt; D-α,β-diaminopropionic acid; D-α-aminobutyric acid; D-α-t-butylglycine; D-(2-thienyl)glycine; D-(3-thienyl)glycine; D-2-aminocaproic acid; D-2-indanylglycine; D-allylglycine.dicyclohexylammonium salt; D-cyclohexylglycine; D-norvaline; D-phenylglycine; β-aminobutyric acid; β-aminoisobutyric acid; (2-bromophenyl)glycine; (2-methoxyphenyl)glycine; (2-methylphenyl)glycine; (2-thiazoyl)glycine; (2-thienyl)glycine; 2-amino-(3-(dimethylamino)-propionic acid; L-α,β-diaminopropionic acid; L-α-aminobutyric acid; L-α-t-butylglycine; L-β-thienyl)glycine; L-2-amino-β-(dimethylamino)-propionic acid; L-2-aminocaproic acid dicyclohexyl-ammonium salt; L-2-indanylglycine; L-allylglycine.dicyclohexyl ammonium salt; L-cyclohexylglycine; L-phenylglycine; L-propargylglycine; L-norvaline; N-α-aminomethyl-L-alanine; D-α,γ-diaminobutyric acid; L-α,γ-diaminobutyric acid; β-cyclopropyl-L-alanine; (N-β-(2,4-dinitrophenyl))-L-α,β-diaminopropionic acid; (N-β-1-(4,4-dimethyl-2,6-dioxocyclohex-1-ylidene)ethyl)-D-α,β-diaminopropionic acid; (N-β-1-(4,4-dimethyl-2,6-dioxocyclohex-1-ylidene)ethyl)-L-α,β-diaminopropionic acid; (N-β-4-methyltrityl)-L-α,β-diaminopropionic acid; (N-β-allyloxycarbonyl)-L-α,β-diaminopropionic acid; (N-γ-1-(4,4-dimethyl-2,6-dioxocyclohex-1-ylidene)ethyl)-D-α,γ-diaminobutyric acid; (N-γ-1-(4,4-dimethyl-2,6-dioxocyclohex-1-ylidene)ethyl)-L-α,γ-diaminobutyric acid; (N-γ-4-methyltrityl)-D-α,γ-diaminobutyric acid; (N-γ-4-methyltrityl)-L-α,γ-diaminobutyric acid; (N-γ-allyloxycarbonyl)-L-α,γ-diaminobutyric acid; D-α,γ-diaminobutyric acid; 4,5-dehydro-L-leucine; cyclopentyl-D-Gly-OH; cyclopentyl-Gly-OH; D-allylglycine; D-homocyclohexylalanine; L-1-pyrenylalanine; L-2-aminocaproic acid; L-allylglycine; L-homocyclohexylalanine; and N-(2-hydroxy-4-methoxy-Bzl)-Gly-OH.

Amino acid analogs include analogs of arginine or lysine. Examples of amino acid analogs of arginine and lysine include, but are not limited to, the following: citrulline; L-2-amino-3-guanidinopropionic acid; L-2-amino-3-ureidopropionic acid; L-citrulline; Lys(Me)₂—OH; Lys(N₃)—OH; Nδ-benzyloxycarbonyl-L-omithine; Nω-nitro-D-arginine; Nω-nitro-L-arginine; α-methyl-omithine; 2,6-diaminoheptanedioic acid; L-omithine; (Nδ-1-(4,4-dimethyl-2,6-dioxo-cyclohex-1-ylidene)ethyl)-D-omithine; (Nδ-1-(4,4-dimethyl-2,6-dioxo-cyclohex-1-ylidene)ethyl) -L-omithine; (Nδ-4-methyltrityl)-D-omithine; (Nδ-4-methyltrityl)-L-omithine; D-omithine; L-omithine; Arg(Me)(Pbf)-OH; Arg(Me)₂—OH (asymmetrical); Arg(Me)₂—OH (symmetrical); Lys(ivDde)-OH; Lys(Me)2—OH.HCl; Lys(Me3)-OH chloride; Nω-nitro-D-arginine; and Nω-nitro-L-arginine.

Amino acid analogs include analogs of aspartic or glutamic acids. Examples of amino acid analogs of aspartic and glutamic acids include, but are not limited to, the following: α-methyl-D-aspartic acid; α-methyl-glutamic acid; α-methyl-L-aspartic acid; γ-methylene-glutamic acid; (N-γ-ethyl)-L-glutamine; [N-α-(4-aminobenzoyl)]-L-glutamic acid; 2,6-diaminopimelic acid; L-α-aminosuberic acid; D-2-aminoadipic acid; D-α-aminosuberic acid; α-aminopimelic acid; iminodiacetic acid; L-2-aminoadipic acid; threo-β-methyl-aspartic acid; γ-carboxy-D-glutamic acid γ,γ-di-t-butyl ester; γ-carboxy-L-glutamic acid γ,γ-di-t-butyl ester; Glu(OAll)-OH; L-Asu(OtBu)—OH; and pyroglutamic acid.

Amino acid analogs include analogs of cysteine and methionine. Examples of amino acid analogs of cysteine and methionine include, but are not limited to, Cys(farnesyl)-OH, Cys(farnesyl)-OMe, α-methyl-methionine, Cys(2-hydroxyethyl)-OH, Cysβ-aminopropyl)-OH, 2-amino-4-(ethylthio)butyric acid, buthionine, buthioninesulfoximine, ethionine, methionine methylsulfonium chloride, selenomethionine, cysteic acid, [2-(4-pyridyl)ethyl]-DL-penicillamine, [2-(4-pyridyl)ethyl]-L-cysteine, 4-methoxybenzyl-D-penicillamine, 4-methoxybenzyl-L-penicillamine, 4-methylbenzyl-D-penicillamine, 4-methylbenzyl-L-penicillamine, benzyl-D-cysteine, benzyl-L-cysteine, benzyl-DL-homocysteine, carbamoyl-L-cysteine, carboxyethyl-L-cysteine, carboxymethyl-L-cysteine, diphenylmethyl-L-cysteine, ethyl-L-cysteine, methyl-L-cysteine, t-butyl-D-cysteine, trityl-L-homocysteine, trityl-D-penicillamine, cystathionine, homocystine, L-homocystine, (2-aminoethyl)-L-cysteine, seleno-L-cystine, cystathionine, Cys(StBu)—OH, and acetamidomethyl-D-penicillamine.

Amino acid analogs include analogs of phenylalanine and tyrosine. Examples of amino acid analogs of phenylalanine and tyrosine include 3-methyl-phenylalanine, 3-hydroxyphenylalanine, α-methyl-3-methoxy-DL-phenylalanine, α-methyl-D-phenylalanine, α-methyl-L-phenylalanine, 1,2,3,4-tetrahydroisoquinoline-3-carboxylic acid, 2,4-dichloro-phenylalanine, 2-(trifluoromethyl)-D-phenylalanine, 2-(trifluoromethyl)-L-phenylalanine, 2-bromo-D-phenylalanine, 2-bromo-L-phenylalanine, 2-chloro-D-phenylalanine, 2-chloro-L-phenylalanine, 2-cyano-D-phenylalanine, 2-cyano-L-phenylalanine, 2-fluoro-D-phenylalanine, 2-fluoro-L-phenylalanine, 2-methyl-D-phenylalanine, 2-methyl-L-phenylalanine, 2-nitro-D-phenylalanine, 2-nitro-L-phenylalanine, 2;4;5-trihydroxy-phenylalanine, 3,4,5-trifluoro-D-phenylalanine, 3,4,5-trifluoro-L-phenylalanine, 3,4-dichloro-D-phenylalanine, 3,4-dichloro-L-phenylalanine, 3,4-difluoro-D-phenylalanine, 3,4-difluoro-L-phenylalanine, 3,4-dihydroxy-L-phenylalanine, 3,4-dimethoxy-L-phenylalanine, 3,5,3′-triiodo-L-thyronine, 3,5-diiodo-D-tyrosine, 3,5-diiodo-L-tyrosine, 3,5-diiodo-L-thyronine, 3-(trifluoromethyl)-D-phenylalanine, 3-(trifluoromethyl)-L-phenylalanine, 3-amino-L-tyrosine, 3-bromo-D-phenylalanine, 3-bromo-L-phenylalanine, 3-chloro-D-phenylalanine, 3-chloro-L-phenylalanine, 3-chloro-L-tyrosine, 3-cyano-D-phenylalanine, 3-cyano-L-phenylalanine, 3-fluoro-D-phenylalanine, 3-fluoro-L-phenylalanine, 3-fluoro-tyrosine, 3-iodo-D-phenylalanine, 3-iodo-L-phenylalanine, 3-iodo-L-tyrosine, 3-methoxy-L-tyrosine, 3-methyl-D-phenylalanine, 3-methyl-L-phenylalanine, 3-nitro-D-phenylalanine, 3-nitro-L-phenylalanine, 3-nitro-L-tyrosine, 4-(trifluoromethyl)-D-phenylalanine, 4-(trifluoromethyl)-L-phenylalanine, 4-amino-D-phenylalanine, 4-amino-L-phenylalanine, 4-benzoyl-D-phenylalanine, 4-benzoyl-L-phenylalanine, 4-bis(2-chloroethyl)amino-L-phenylalanine, 4-bromo-D-phenylalanine, 4-bromo-L-phenylalanine, 4-chloro-D-phenylalanine, 4-chloro-L-phenylalanine, 4-cyano-D-phenylalanine, 4-cyano-L-phenylalanine, 4-fluoro-D-phenylalanine, 4-fluoro-L-phenylalanine, 4-iodo-D-phenylalanine, 4-iodo-L-phenylalanine, homophenylalanine, thyroxine, 3,3-diphenylalanine, thyronine, ethyl-tyrosine, and methyl-tyrosine.

Amino acid analogs include analogs of proline. Examples of amino acid analogs of proline include, but are not limited to, 3,4-dehydro-proline, 4-fluoro-proline, cis-4-hydroxy-proline, thiazolidine-2-carboxylic acid, and trans-4-fluoro-proline.

Amino acid analogs include analogs of serine and threonine. Examples of amino acid analogs of serine and threonine include, but are not limited to, 3-amino-2-hydroxy-5-methylhexanoic acid, 2-amino-3-hydroxy-4-methylpentanoic acid, 2-amino-3-ethoxybutanoic acid, 2-amino-3-methoxybutanoic acid, 4-amino-3-hydroxy-6-methylheptanoic acid, 2-amino-3-benzyloxypropionic acid, 2-amino-3-benzyloxypropionic acid, 2-amino-3-ethoxypropionic acid, 4-amino-3-hydroxybutanoic acid, and α-methylserine.

Amino acid analogs include analogs of tryptophan. Examples of amino acid analogs of tryptophan include, but are not limited to, the following: α-methyl-tryptophan; β-(3-benzothienyl)-D-alanine; β-(3-benzothienyl)-L-alanine; 1-methyl-tryptophan; 4-methyl-tryptophan; 5-benzyloxy-tryptophan; 5-bromo-tryptophan; 5-chloro-tryptophan; 5-fluoro-tryptophan; 5-hydroxy-tryptophan; 5-hydroxy-L-tryptophan; 5-methoxy-tryptophan; 5-methoxy-L-tryptophan; 5-methyl-tryptophan; 6-bromo-tryptophan; 6-chloro-D-tryptophan; 6-chloro-tryptophan; 6-fluoro-tryptophan; 6-methyl-tryptophan; 7-benzyloxy-tryptophan; 7-bromo-tryptophan; 7-methyl-tryptophan; D-1,2,3,4-tetrahydro-norharman-3-carboxylic acid; 6-methoxy-1,2,3,4-tetrahydronorharman-1-carboxylic acid; 7-azatryptophan; L-1,2,3,4-tetrahydro-norharman-3-carboxylic acid; 5-methoxy-2-methyl-tryptophan; and 6-chloro-L-tryptophan.

In some embodiments, amino acid analogs are racemic. In some embodiments, the D isomer of the amino acid analog is used. In some embodiments, the L isomer of the amino acid analog is used. In other embodiments, the amino acid analog comprises chiral centers that are in the R or S configuration. In still other embodiments, the amino group(s) of a β-amino acid analog is substituted with a protecting group, e.g., tert-butyloxycarbonyl (BOC group), 9-fluorenylmethyloxycarbonyl (FMOC), tosyl, and the like. In yet other embodiments, the carboxylic acid functional group of a β-amino acid analog is protected, e.g., as its ester derivative. In some embodiments the salt of the amino acid analog is used.

A “non-essential” amino acid residue is a residue that can be altered from the wild-type sequence of a polypeptide without abolishing or substantially abolishing its essential biological or biochemical activity (e.g., receptor binding or activation). An “essential” amino acid residue is a residue that, when altered from the wild-type sequence of the polypeptide, results in abolishing or substantially abolishing the polypeptide's essential biological or biochemical activity.

A “conservative amino acid substitution” is one in which the amino acid residue is replaced with an amino acid residue having a similar side chain. Families of amino acid residues having similar side chains have been defined in the art. These families include amino acids with basic side chains (e.g., K, R, H), acidic side chains (e.g., D, E), uncharged polar side chains (e.g., G, N, Q, S, T, Y, C), nonpolar side chains (e.g., A, V, L, I, P, F, M, W), beta-branched side chains (e.g., T, V, I) and aromatic side chains (e.g., Y, F, W, H). Thus, a predicted nonessential amino acid residue in a polypeptide, for example, is replaced with another amino acid residue from the same side chain family. Other examples of acceptable substitutions are substitutions based on isosteric considerations (e.g. norleucine for methionine) or other properties (e.g., 2-thienylalanine for phenylalanine).

The term “polypeptide” refers to a linear organic polymer consisting of a large number of amino-acid residues bonded together in a chain, forming part of (or the whole of) a protein molecule.

The term “α-polypeptide” refers to are polypeptides derived from α-amino acids.

The term “β-polypeptide” refers to are polypeptides derived from β-amino acids.

The term “aliphatic” or “aliphatic group” refers to a hydrocarbon moiety that may be straight-chain (i.e., unbranched), branched, or cyclic (including fused, bridging, and spiro-fused polycyclic) and may be completely saturated or may contain one or more units of unsaturation. Suitable aliphatic groups include, but are not limited to, linear or branched, alkyl, alkenyl, and alkynyl groups, and hybrids thereof. As used herein the terms “aliphatic” or “aliphatic group”, also encompass partially substituted analogs of these moieties where at least one of the hydrogen atoms of the aliphatic group is replaced by an atom that is not carbon or hydrogen.

The term “linker” refers to a chemical group that connects one or more other chemical groups via at least one covalent bond.

While the invention has been described with reference to an exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiments disclosed as the best mode contemplated for carrying out this invention, but that the invention will include all embodiments falling within the scope of the appended claims. Any combination of the described elements in all possible variations thereof is encompassed by the invention unless otherwise indicated herein or otherwise clearly contradicted by context.

### Janus Base Nanotubes

Self-assembled nanomaterials of the present disclosure comprise Janus base nanotubes.

In some embodiments, the Janus base nanotube comprises a compound of Formula (I):

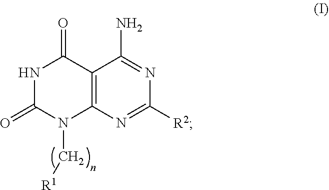

or a pharmaceutically acceptable salt thereof, wherein:

n is 1, 2, 3, 4, 5, or 6;

R¹, R⁵, R¹¹ and R¹⁵ are each independently selected from an α-amino acid, a β-amino acid, an α-polypeptide, and a β-polypeptide,

R², R⁶ and R⁶ are each independently selected from H, CH₃, and NHR^{z}; and

R^{z}, R¹² and R¹⁶ are each independently H or a C₁ to C₂₀ aliphatic group.

In some embodiments, the Janus base nanotube comprises a compound of Formula (III):

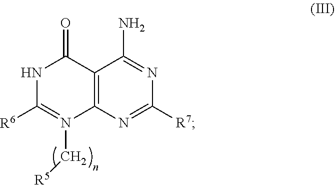

or a pharmaceutically acceptable salt thereof, wherein:

n is 1, 2, 3, 4, 5, or 6;

R¹, R⁵, R¹¹ and R¹⁵ are each independently selected from an α-amino acid, a β-amino acid, an α-polypeptide, and a β-polypeptide,

R², R⁶ and ⁷ are each independently selected from H, CH₃, and NHR^{z}; and

R^{z}, R¹² and R¹⁶ are each independently H or a C₁ to C₂₀ aliphatic group.

In some embodiments, the Janus base nanotube comprises a compound of Formula (V):

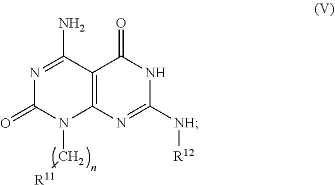

or a pharmaceutically acceptable salt or ester thereof, wherein:

n is 1, 2, 3, 4, 5, or 6;

R¹, R⁵, R¹¹ and R¹⁵ are each independently selected from an α-amino acid, a β-amino acid, an α-polypeptide, and a β-polypeptide,

R², R⁶ and R⁷ are each independently selected from H, CH₃, and NHR^{z}; and

R^{z}, R¹² and R¹⁶ are each independently H or a C₁ to C₂₀ aliphatic group.

In some embodiments, the Janus base nanotube comprises a compound of Formula (VII):

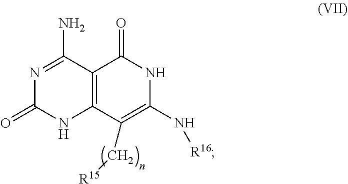

or a pharmaceutically acceptable salt or ester thereof, wherein:

n is 1, 2, 3, 4, 5, or 6;

R¹, R⁵, R¹¹ and R¹⁵ are each independently selected from an α-amino acid, a β-amino acid, an α-polypeptide, and a β-polypeptide,

R², R⁶ and R⁷ are each independently selected from H, CH₃, and NHR^{z}; and

R^{z}, R¹² and R¹⁶ are each independently H or a C₁ to C₂₀ aliphatic group.

In some embodiments, the Janus base nanotube comprises a compound of Formula (II):

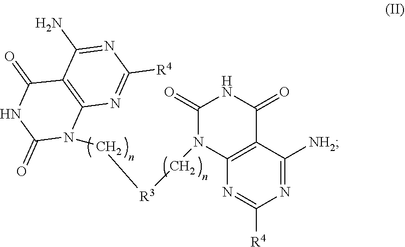

or a pharmaceutically acceptable salt thereof, wherein:

n is 1, 2, 3, 4, 5, or 6;

R³, R^{8,} R¹³ and R¹⁷ are each independently selected from an α-amino acid, a β-amino acid, an α-polypeptide, and a β-polypeptide;

R⁴, R⁹ and R¹⁰ are each independently H, CH₃, or NHR^{z}; and

R^{z}, R¹⁴, R¹⁸ are each independently H or a C₁ to C₂₀ aliphatic group.

In some embodiments, the Janus base nanotube comprises a compound of Formula (IV):

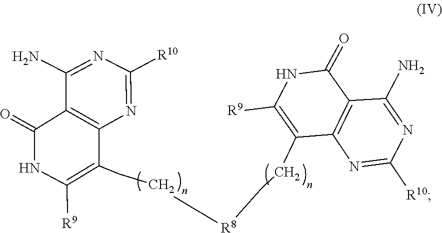

or a pharmaceutically acceptable salt thereof, wherein

n is 1, 2, 3, 4, 5, or 6;

R³, R^{8,} R¹³ and R¹⁷ are each independently selected from an α-amino acid, a β-amino acid, an α-polypeptide, and a β-polypeptide;

R⁴, R⁹ and R¹⁰ are each independently H, CH₃, or NHR^{z}; and

R^{z}, R¹⁴, R¹⁸ are each independently H or a C₁ to C₂₀ aliphatic group.

In some embodiments, the Janus base nanotube comprises a compound of Formula (VI):

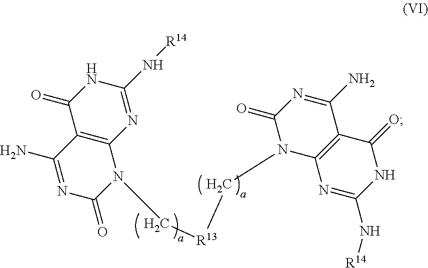

or a pharmaceutically acceptable salt thereof, wherein

n is 1, 2, 3, 4, 5, or 6;

R³, R^{8,} R¹³ and R¹⁷ are each independently selected from an α-amino acid, a β-amino acid, an α-polypeptide, and a β-polypeptide;

R⁴, R⁹ and R¹⁰ are each independently H, CH₃, or NHR^{z}; and

R^{z}, R¹⁴, R¹⁸ are each independently H or a C₁ to C₂₀ aliphatic group.

In some embodiments, the Janus base nanotube comprises a compound of Formula (VIII):

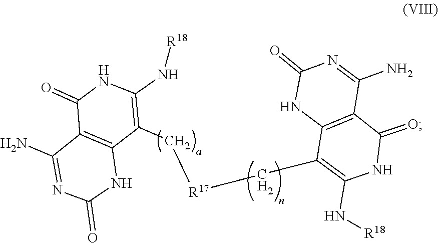

or a pharmaceutically acceptable salt thereof, wherein:

n is 1, 2, 3, 4, 5, or 6;

R³, R^{8,} R¹³ and R¹⁷ are each independently selected from an α-amino acid, a β-amino acid, an α-polypeptide, and a β-polypeptide;

R⁴, R⁹ and R¹⁰ are each independently H, CH₃, or NHR^{z}; and

R^{z}, R¹⁴, R¹⁸ are each independently H or a C₁ to C₂₀ aliphatic group.

In some embodiments, the Janus base nanotube comprises a compound of Formula (IX),

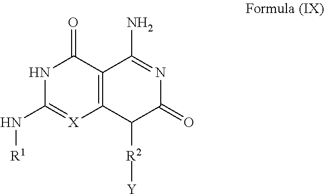

or a pharmaceutically acceptable salt, wherein:

X is CH or nitrogen;

R₂ is hydrogen or a C₁ to C₂₀ linker group;

Y is absent when R₂ is hydrogen, or is an amino acid or polypeptide having an amino group covalently bound to an α-carbon of the amino acid and the amino group is covalently bound to the linker group R²; and

R₁ is hydrogen or C₁ to C₂₀ aliphatic moiety, such as alkyl, straight or branched chain, saturated or unsaturated alkyl.

In some embodiments, the Janus base nanotube comprises a compound of Formula (XI),

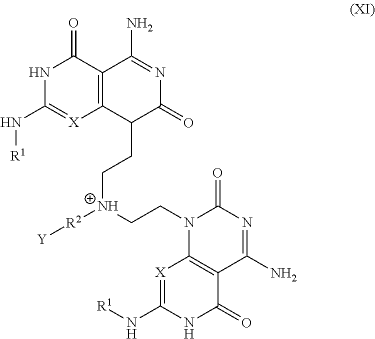

or a pharmaceutically acceptable salt, wherein:

X is CH or nitrogen;

R₂ is hydrogen or a C₁ to C₂₀ linker group;

Y is absent when R² is hydrogen, or is an amino acid or polypeptide having an amino group covalently bound to an α-carbon of the amino acid and the amino group is covalently bound to the linker group R²; and

R¹ is hydrogen or a C1 to C20 aliphatic moiety, such as alkyl, straight or branched chain, saturated or unsaturated.

### Self-Assembled Nanomaterial

Disclosed herein is a self-assembled nanomaterial comprising a Janus base nanotube having a biologically active molecule noncovalently adhered thereto, wherein the biologically active molecule comprises an extracellular matrix (ECM) molecule, a bioactive molecule, or a combination thereof. In an aspect, the NM is in the form of fibrils, having an average diameter of 50 nm to 2 mm, and an average length of 100 nm to 100 mm. In a preferred aspect, the ECM molecule is assembled non-covalently.

While not limited to this aspect, the ratio of JBNTs to ECM molecule is 1000:1 to 1:1.

As used herein, a single compartment nanomaterial includes a single population of self-assembled nanomaterials, that is, a single type of JBNT and one or more ECM molecules adhering to the JBNT.

As used herein, a multiple compartment nanomaterial includes two or more populations of self-assembled nanomaterials that form a multi-compartmental structure, through electrostatic layer-by-layer assembly, for example. In an example, a first population of JBNTs with TGF-β can be fabricated inside a second population of JBNTs with Matn3. For example. Opposite electrostatic charges on the first and second populations of JBNTs can drive assembly of the multiple compartment nanomaterial. By assembling the first and second populations in order, the one population will form the interior compartment and the second population will form the exterior compartment.

As used herein, injectable can mean injectable through a needle having a diameter of 0.1 mm to 10 mm. In an aspect, an assembled nanomaterial is injected. In another aspect, precursors for the nanomaterial are injected and self-assemble in vivo.

Exemplary ECM molecules that may be used in the disclosed nanomaterials include hydroxyapatite, fibronectin, Matn1, MAtn3, laminin, a collagen (e.g., type I collagen, type II collagen), elastin, vitronectin, fibrillin, perlecan, fibrinogen, osteonectin, tenascin, thrombospondin, an intercellular adhesion molecule (ICAM1-5), an integrin, a proteoglycan (aggrecan, a glycosaminoglycan (e.g., hyaluronic acid, chondroitin sulfate, dermatin sulfate, keratan sulfate, heparin, heparin sulfate), a glycoprotein, and combinations thereof.

Exemplary bioactive molecules that may be used in the disclosed nanomaterials include TGFβ, VEGF, IGF, EGF, PDGF, a BMPs, an FGF, GDNF, HGF, PGF, NGF, TNF-α, SDF-1, dexamethasone, an siRNA, an miRNA, a growth factor, a small-molecule drug, and combinations thereof.

### Cell Growth and Tissue Regeneration

Advantageously, the materials described herein are tunable materials comprising Janus base nanotubes and extracellular matrix molecules (ECMs). The JBNTs can assemble with different ECMs to form different NMs for different cells/tissues. In addition, the NMs can be fabricated with multi-functional layers or compartments to achieve various functions more than supporting cell growth (such as drug release).

For tissue regeneration, the NMs can include Matn3 for growth plate fracture repair. However, other ECM molecules can be used for brain regeneration growth plate repair, and other applications.

### Drug Delivery

Various biomaterial scaffolds have been developed for stem cells anchorage and functions to generate tissue constructs for in vitro and in vivo uses. Growth factors are typically applied to the scaffolds to mediate cell differentiation. Conventionally, growth factors were not strictly localized in the scaffolds; thus, they may leak into the surrounding environment, causing undesired side effects on tissues or cells. Hence, there is a need for improved tissue construct strategies based on highly localized drug delivery and homeostatic microenvironment.

Disclosed herein are injectable nano-matrix (NM) scaffold with a layer-by-layer structure inside the nano-sized fibers of their scaffolds based on the controlled self-assembly at the molecular level. The NM was hierarchically assembled from Janus base nanotubes (JBNTs), matrilin-3, and transforming growth factor beta-1 (TGF-β1) via bioaffinity. JBNTs, which formed the NM backbone, are novel DNA-inspired nanomaterials that mimic the natural helical nanostructures of collagens. The chondrogenic factor, TGF-β1, was enveloped in the inner layer inside the NM fibers to prevent its release. Matrilin-3 was incorporated into the outer layer to create a cartilage-mimicking microenvironment and to maintain tissue homeostasis. In some embodiments, human mesenchymal stem cells (hMSCs) had a strong preference to anchor along the NM fibers and formed a localized homeostatic microenvironment. In preferred embodiments, this NM generated highly organized structures via molecular self-assembly and achieved localized drug delivery and stem cell anchorage for homeostatic tissue constructs.

### Pharmaceutical Compositions

Injectable compositions comprise the self-assembled nanomaterials described above and a pharmaceutically acceptable carrier. The self-assembled nanomaterials may o be administered parenterally in a sterile medium, either subcutaneously, or intravenously, or intramuscularly, or intrasternally, or by infusion techniques, in the form of sterile injectable aqueous or oleaginous suspensions. Advantageously, adjuvants such as a local anaesthetic, preservative and buffering agents can be dissolved in the vehicle.

### Tissue Engineering

A method of tissue engineering comprises injecting into a tissue the injectable composition described herein. Exemplary tissues are selected from cartilage, bone, brain, spine, joint, nerve, ligament and tendon, bone marrow, heart, eye, liver, kidney, and lung.

### EXEMPLIFICATION

### Example 1

### Formulation of an NM Based on JBNTs and Matn1

This example demonstrates the development of the formulation of an NM based on JBNTs and Matn1.

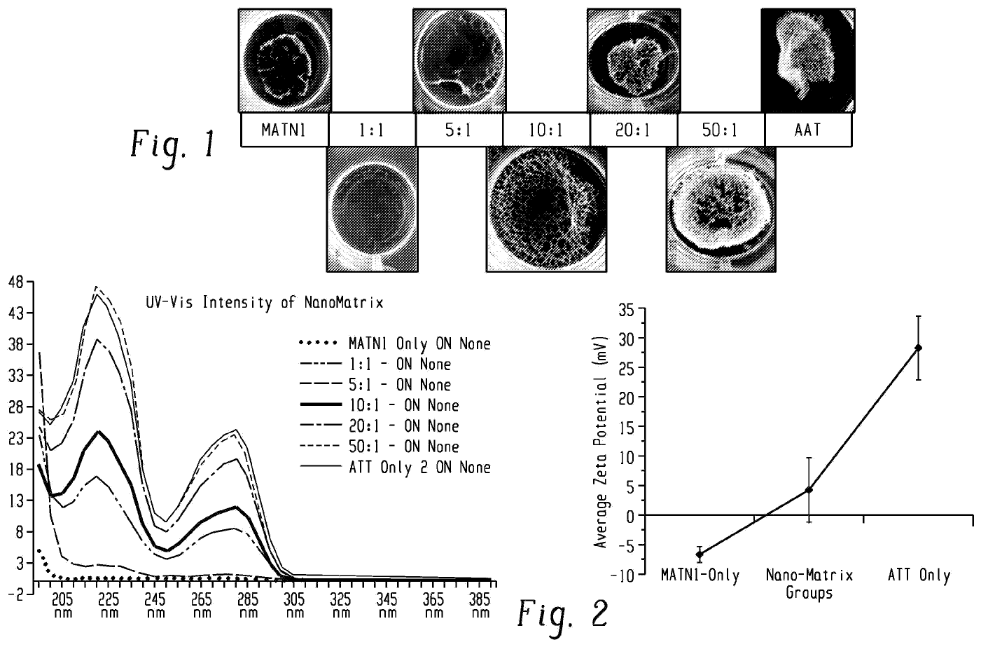

At the 10:1 ratio, JBNTs and Matn1 can assemble into a solid scaffold in water without adding chemical initiators or UV light. Camera images showed the macro-structures of NMs in FIG. 1.

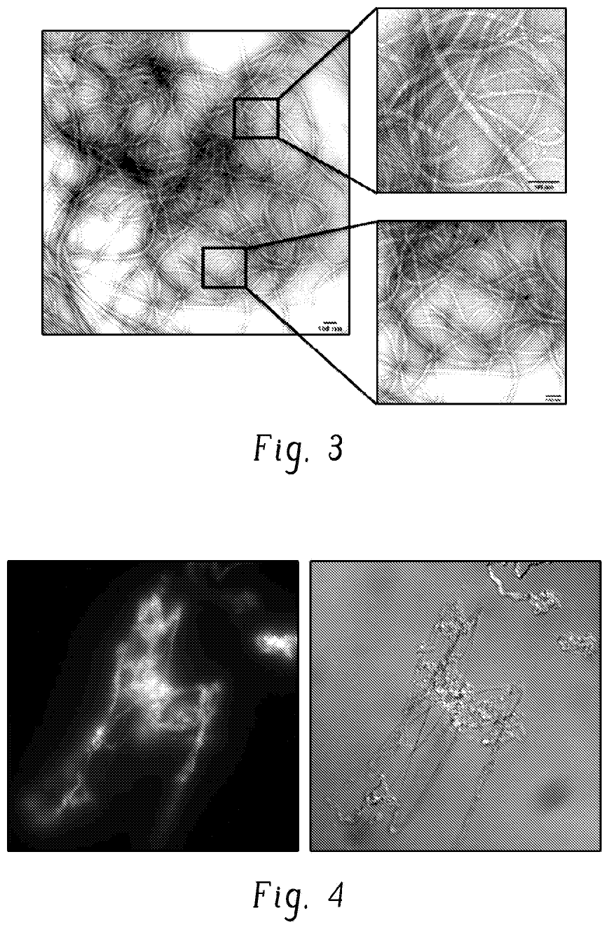

UV-Vis and zeta potential were conducted to show the incorporation of JBNTs and Matn1 (FIG. 2).

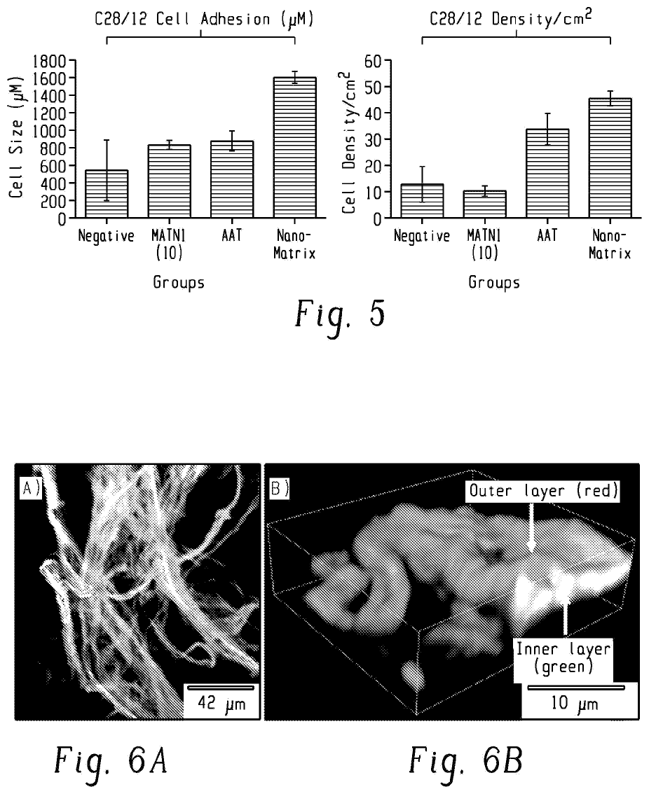

TEM images showed the nanostructures of the NM in FIG. 3.

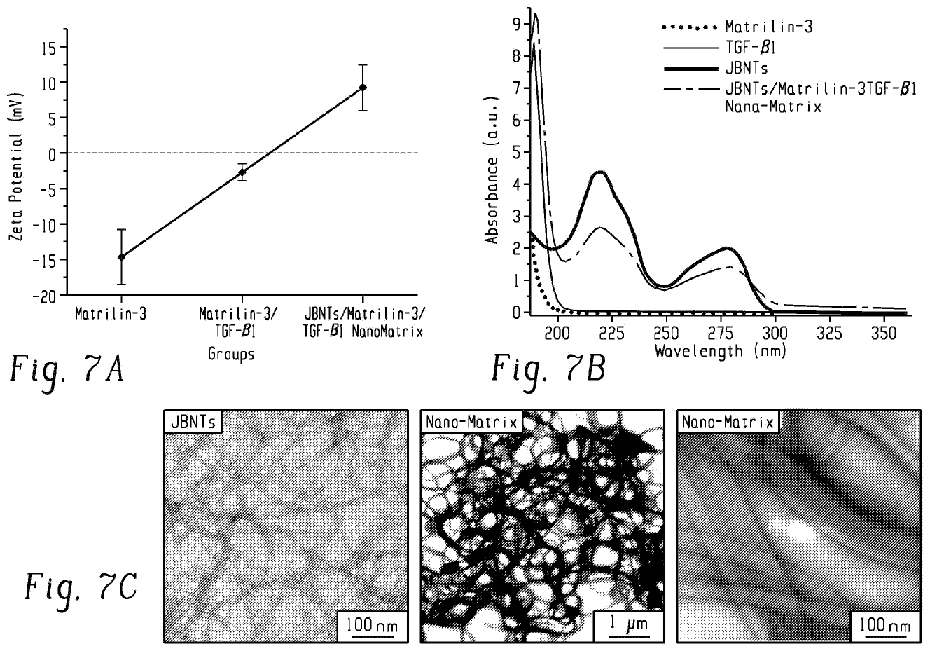

Widefield images showed the micro-structures of the NM in FIG. 4.

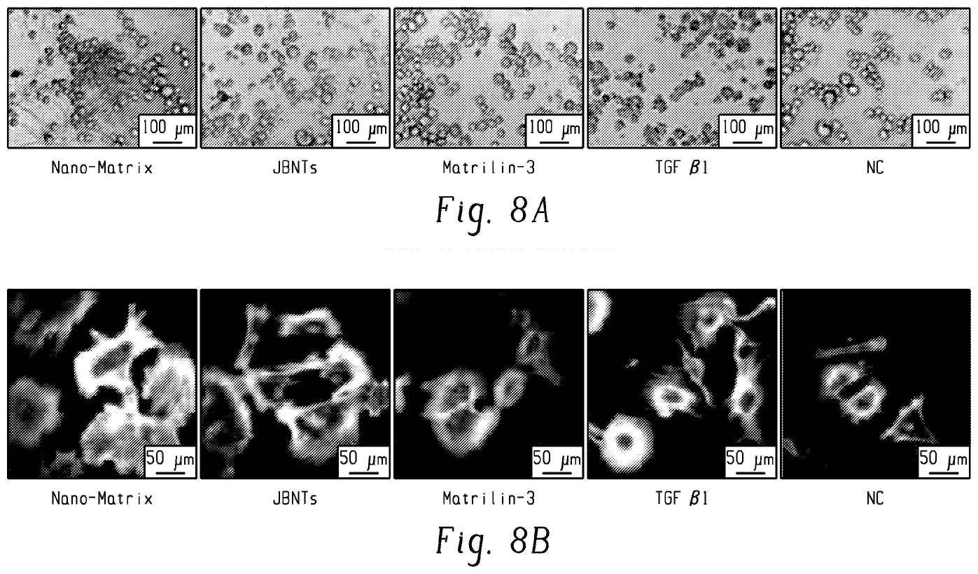

FIG. 5 are graphs showing cell adhesion and density on the NM.

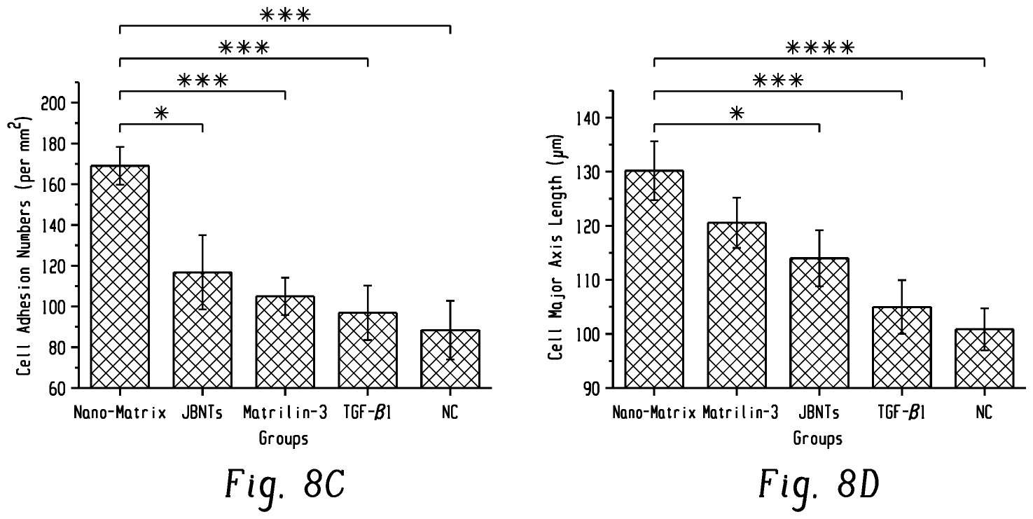

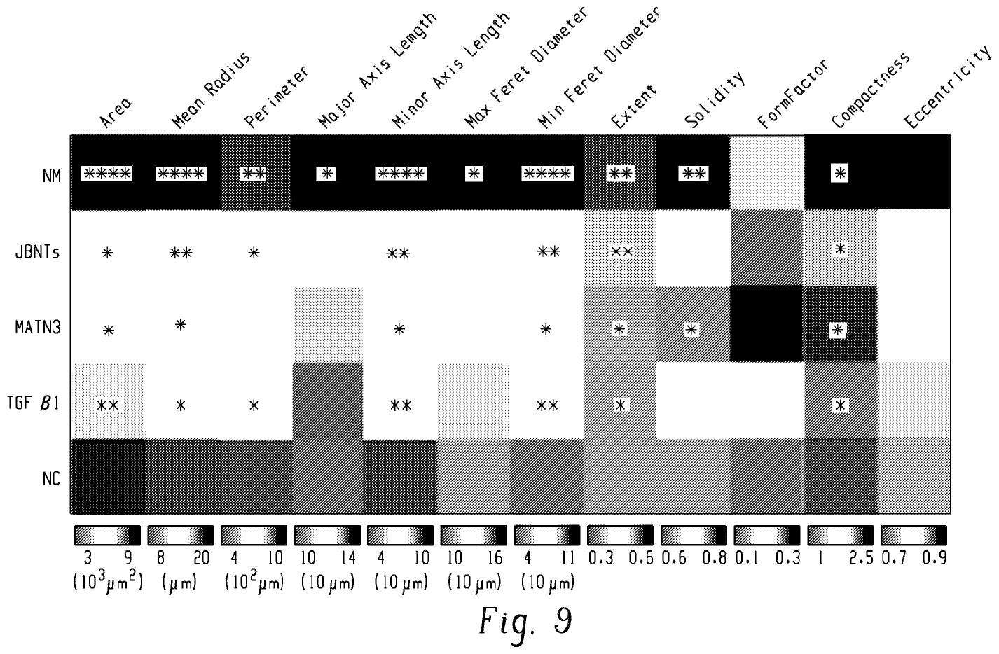

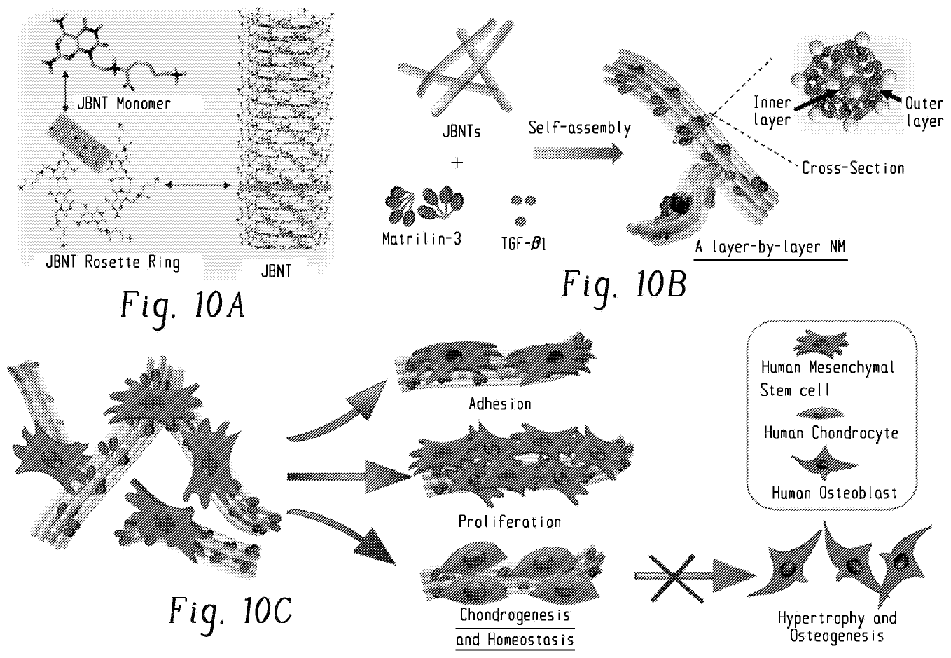

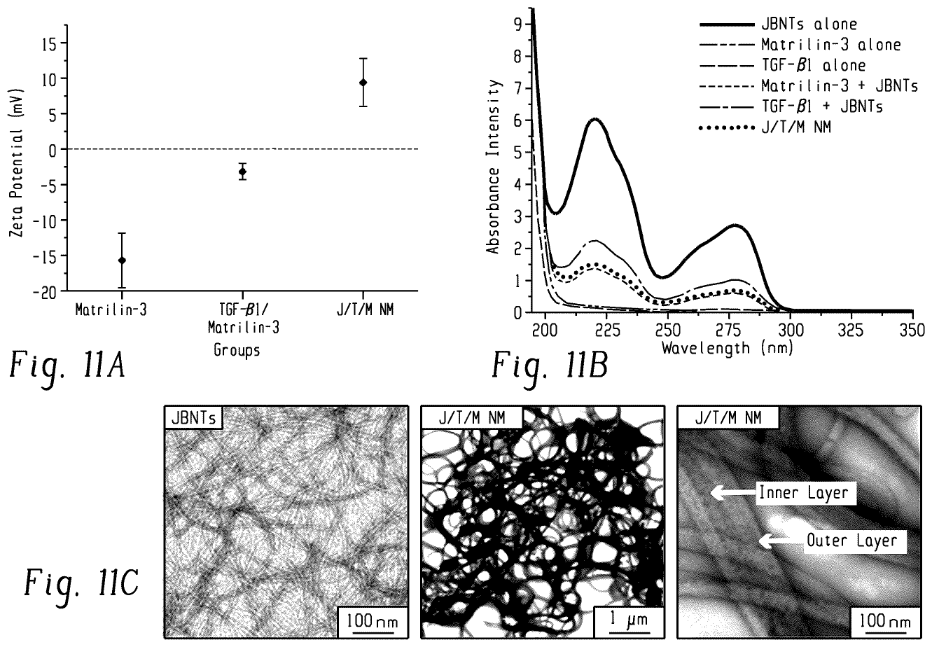

NMs with multiple layers and multiple compartments were also developed. For example, a double-layered NM from JBNTs, Matn3 and TGFβ was also created. (TGFβ is a growth factor drug can promote chondrogenesis.) The layer with TGFβ is fabricated inside of the JBNT/Matn3 layer (FIG. 6). Zeta potential, UV-vis and TEM analysis demonstrated the incorporation of JBNTs, Matn3 and TGFβ (FIG. 7). Cell adhesion results demonstrated the multi-layered NM can further improve cell functions (FIGS. 8 and 9).

### Example 2

### Controlled Self-Assembly of DNA-Mimicking Nanotubes to Form a Layer-by-Layer Scaffold for Homeostatic Tissue Constructs

In this example, an injectable nano-matrix (NM) scaffold with a layer-by-layer structure were developed inside its nano-sized fiber of the scaffold based on the controlled self-assembly at the molecular level. This NM successfully generated highly organized structures via molecular self-assembly and achieved localized drug delivery and stem cell anchorage for homeostatic tissue constructs.

### Results and Discussion

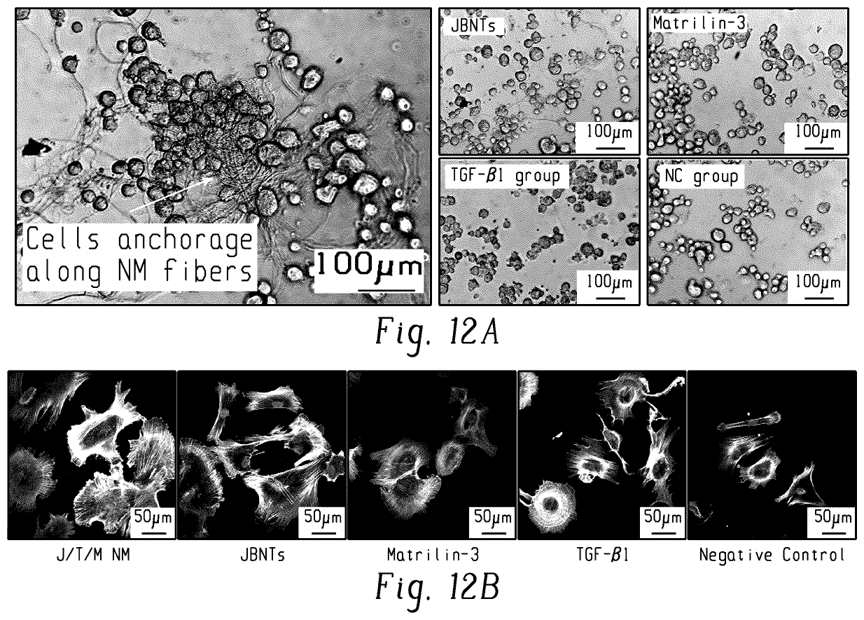

Because JBNTs are different from conventional material fibers produced electrospinning or 3D print. The chemical structure of JBNTs is shown in FIG. 10A. Each JBNT fiber has a fixed molecular structure with a diameter of 3.5 nm. Proteins (such as TGF-β1 and matrilin3) were incorporated between JBNTs. In other words, multiple JBNTs “sandwiched” TGF-β1 or matrilin3 inside their bundles (as shown in FIG. 10B). When JBNTs, TGF-β1 and matrilin3 formed a layer-by-layer NM, the inner layer is bundles of JBNTs enveloped TGF-β1, and the outer layer are JBNT bundles enveloped matrilin3. Such layer-by-layer structure was characterized and determined by the experiments below.

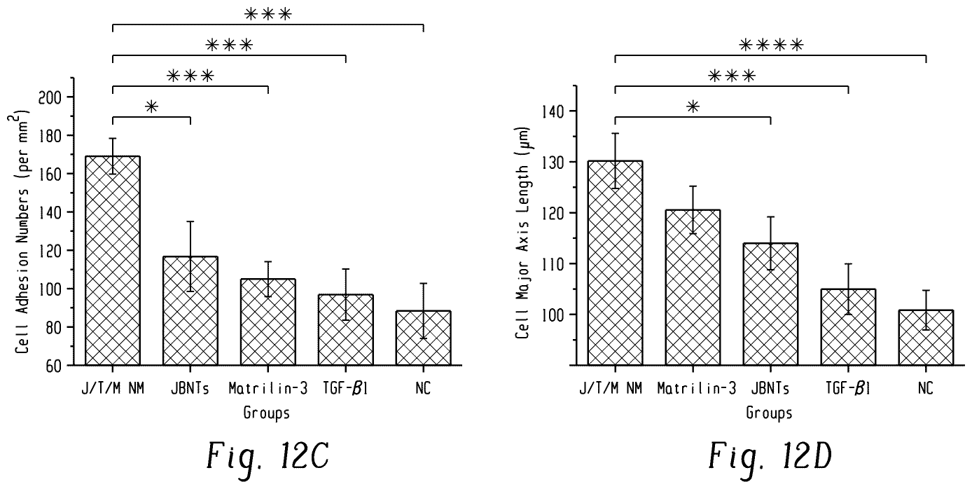

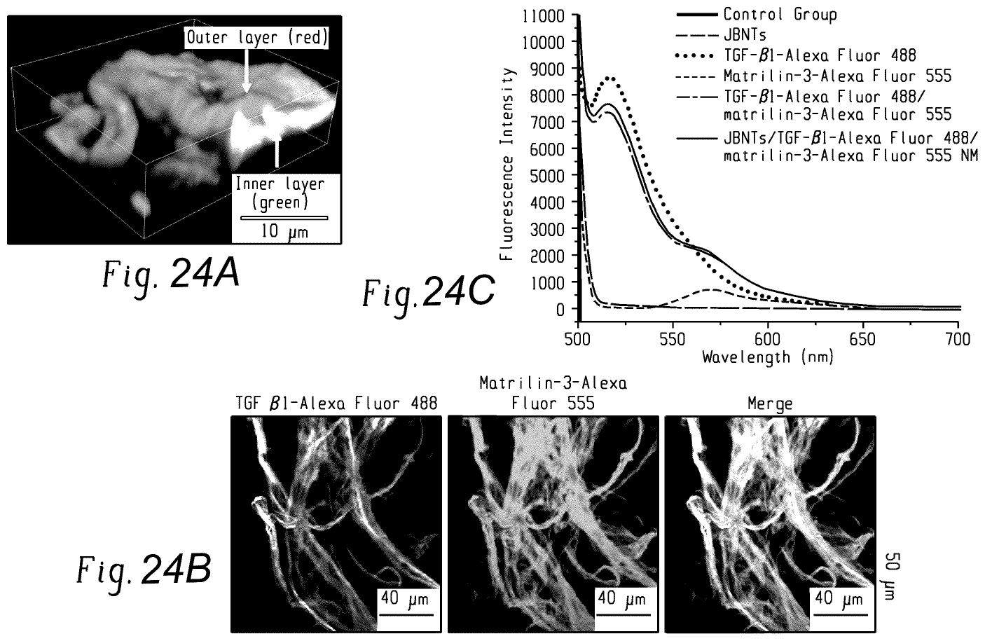

Under physiological conditions, matrilin-3 is negatively charged based on its isoelectric point. As shown in FIG. 11A, the zeta-potential of matrilin-3 was approximately −15 mV. When mixed with TGF-β1, the zeta-potential of the solution increased to an approximately neutral value, indicating the combination of the two proteins via charge interactions. The fluorescence resonance energy transfer (FRET) between matrilin-3 and TGF-β1 also confirmed this binding, as shown in FIG. 24B. Because the isoelectric point of lysine is 9.74, JBNTs are positively charged in physiological environments. The TGF-β1/matrilin-3 complex can further bind with JBNTs, resulting in a charge reversal from the negative/neutral charge of the TGF-β1/matrilin-3 complex to a positive charge for the J/T/M complex, as shown in FIG. 11A. This result demonstrates the orderly assembly of JBNTs, TGF-β1, and matrilin-3 into a J/T/M NM consisting of all three components.

Furthermore, the UV-Vis spectra characterized the assembly among the JBNTs, TGF-β1, and matrilin-3 and clarified the formation of the hierarchical layer-by-layer interior structure of the J/T/M NM. JBNTs mimic collagens in terms of their fibrous morphology and lysine surface chemistry. Although both TGF-β1 and matrilin-3 can interact with JBNTs, similar to their natural binding with collagens, the binding affinity between JBNTs and the two proteins are different. JBNTs have two absorption peaks at 220 nm and 280 nm, which are considered to arise from the lysine side chain and the aromatic rings of the Janus bases, respectively. When the JBNTs were mixed with TGF-β1, the two JBNT peaks decreased, indicating binding between TGF-β1 and the JBNTs (FIG. 11B). When the JBNTs were mixed with matrilin-3, a more obvious decrease in the absorption peaks occurred, indicating more significant binding between matrilin-3 and the JBNTs (FIG. 11B). JBNTs were added to incorporate with the TGF-β1/matrilin-3 complex. For the assembled J/T/M NM, the absorption curve was located between those of the TGF-β1/JBNT mixture and the matrilin-3/JBNT mixture, demonstrating the coexistence of these two types of bonds between the JBNTs and the proteins (FIG. 11B). Moreover, the J/T/M NM curve is much closer to the matrilin-3/JBNT curve than the TGF-β1/JBNT curve; thus, the major interaction in the J/T/M NM occurs between the JBNTs and matrilin-3. Because abundant JBNTs were added to the NM after the assembly of TGF-β1 and matrilin-3, it is clear that the JBNTs preferentially bind with matrilin-3 and form a layer-by-layer structure with matrilin-3 in the outer layer and TGF-β1 in the inner layer. This layer-by-layer structure was confirmed by TEM (FIG. 11C) and fluorescence microscopy (FIGS. 24A-24B).

In data not shown, fluorescence spectra and confocal images of J/T/M NM formed with JBNTs and fluorescently labeled proteins include 3D confocal images of JBNTs/TGF-β1-Alex Fluor® 488/matrilin-3-Alex Fluor® 555 NM; and 2D confocal images of JBNTs/TGF-β1-Alex Fluor® 488/matrilin-3-Alex Fluor® 555 NM in different channels. The FRET process between fluorescent dye-labeled proteins was characterized by the fluorescence spectra.

TEM was performed to characterize the morphology of the JBNTs and the J/T/M NM. As shown in FIG. 11C, the JBNTs consisted of thin individual nanotubes with a diameter of approximately 3.5 nm. When the JBNTs were combined with the proteins, the thick bundles of JBNTs and protein formed a scaffold structure. The scaffold morphologically and biologically mimics the cartilage ECM, which can provide anchor sites and bioactive molecules for a pro-chondrogenic, anti-hypertrophy microenvironment. Importantly, the J/T/M NM is not a simple mixture of the three components. Instead, the NM consists of two layers (outer layer and inner layer) and each layer consisted bundles of JBNTs (FIG. 11C, J/T/M NM). FIG. 24A demonstrated a cross section of the NM. The red-fluorescence-labeled matrilin-3 enveloped in JBNT bundles (confirmed by the UV-vis and TEM results) formed an outer layer, thus providing an optimal microenvironment for cartilage because of the enhanced stem cell anchorage of the JBNTs and the anti-hypertrophy property of matrilin-3. Moreover, the green-fluorescence-labeled TGF-β1 enveloped in JBNT bundles (confirmed by the UV-vis and TEM results) formed an inner layer, generating an inner layer to store growth factors and allowing the TGF-β1 to be localized in the NM instead of leaking into the surrounding environment or undesired locations. In this manner, the TGF-β1 is bioactive when cells grow on the NM, as demonstrated below by cell function experiments.

To further verify the structure of the J/T/M NM, fluorescence spectral analysis was performed. As mentioned above, TGF-β1 and matrilin-3 were labeled with the fluorescent dyes Alexa Fluor® 488 and Alexa Fluor® 555, respectively. All sample groups were excited with a 488-nm laser. As shown in FIG. 24C, no emission peaks arose for the JBNTs or the control group. Emission peaks occurred at approximately 520 and 570 nm for the TGF-β1-Alexa Fluor® 488 and matrilin-3-Alexa Fluor® 555 groups, respectively. When TGF-β1-Alexa Fluor® 488 and matrilin-3-Alexa Fluor® 555 were combined, FRET occurred between the two fluorescent dyes. The TGF-β1-Alexa Fluor® 488 group serves as a donor while the matrilin-3-Alexa Fluor® 555 group serves as an acceptor, which reduces the emission peak at 520 nm and increases the emission peak at 570 nm. The FRET phenomenon indicates that matrilin-3 and TGF-β1 are sufficiently close (<10 nm) because it is a distance-dependent physical process. These results provide strong additional evidence demonstrating the assembly of TGF-β1 and matrilin-3 during the formation of the J/T/M NM.

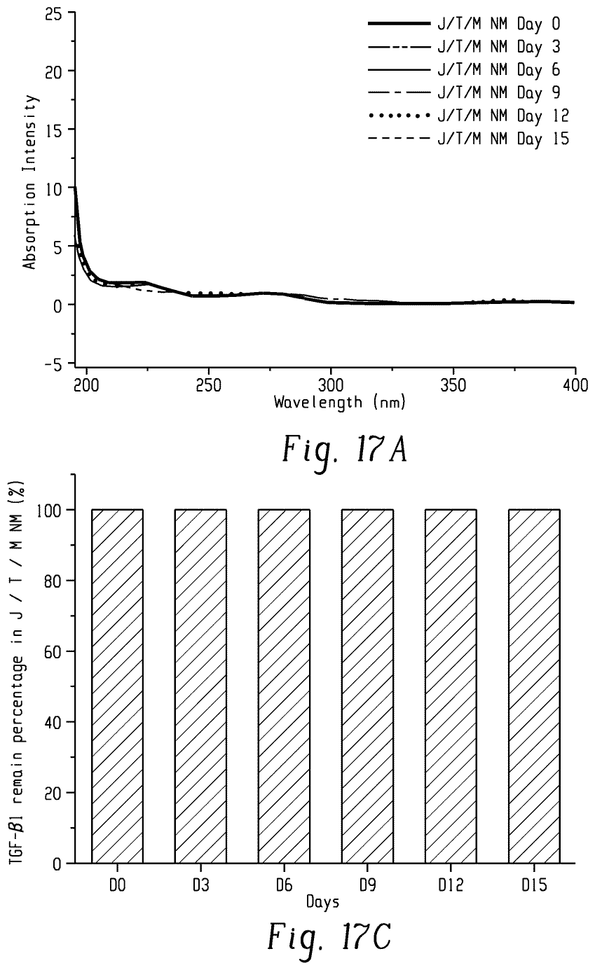

The structural stability of the J/T/M NM and whether the layer-by-layer structure can prevent leakage of TGF-β1. As shown in FIGS. 17A-17B, there is no change of the UV-vis absorption curves of the J/T/M NM in at least 15 days in water. Such result demonstrated that the J/T/M NM had an excellent structure stability in water and did not disassemble in at least two weeks. (If the J/T/M NM disassembled, its UV-vis absorption would increase significantly.) Moreover, consistence with the results above, we also demonstrated that there was minimal leakage of TGF-β1 from the NM after 15 days in a phosphate buffered saline (PBS) buffer. Therefore, the J/T/M NM can maintain its layer-by-layer structure very well and prevented leakage of TGF-β1.

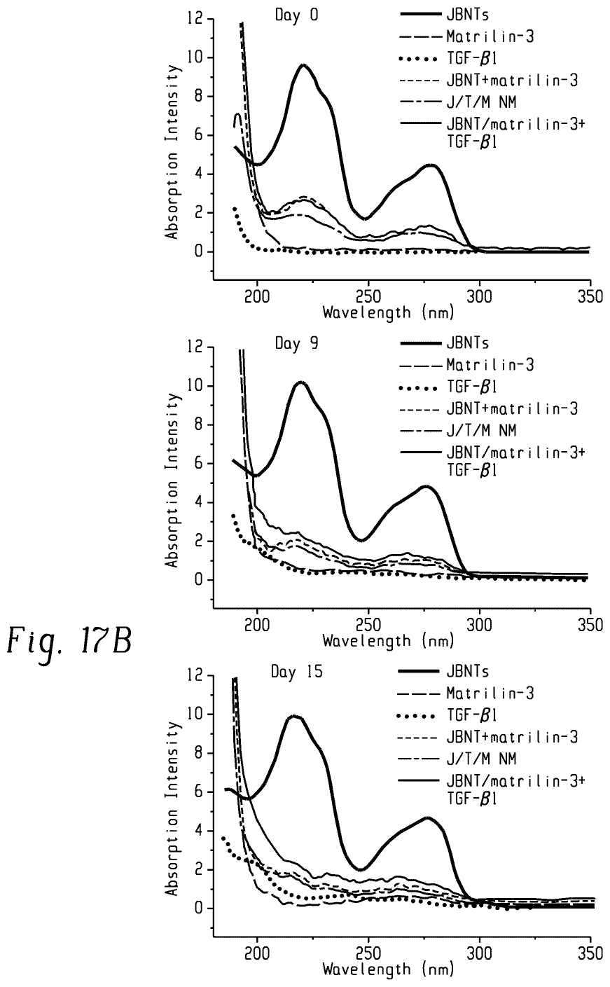

The J/T/M NM is an injectable solid scaffold whose formation undergoes a rapid biomimetic process. As shown in FIG. 18, JBNTs were pipetted into the protein solution in a physiological environment (water solution, no UV light, no chemical additives, and no heating). In less than 30 s, the solid white mesh-like NM scaffold was formed. The assembly occurs as follows: 1) positively charged JBNTs present an electrostatic attraction to the negatively charged matrilin-3; 2) JBNTs mimic collagens and naturally bind to TGF-β1. Because the assembled scaffold is structurally flexible (perhaps due to its DNA-mimicking nanotubular backbones), it can pass through a pipette tip. Therefore, the J/T/M NM has great potential for intralesional injection into irregularly shaped defects. It was noted that conventional injectable hydrogels differ from the disclosed NM scaffold, as conventional hydrogels are homogenous, semisolid materials. In contrast, the disclosed NM scaffold is a porous, solid material with fibril structures. Very few injectable scaffolds have been developed thus far.

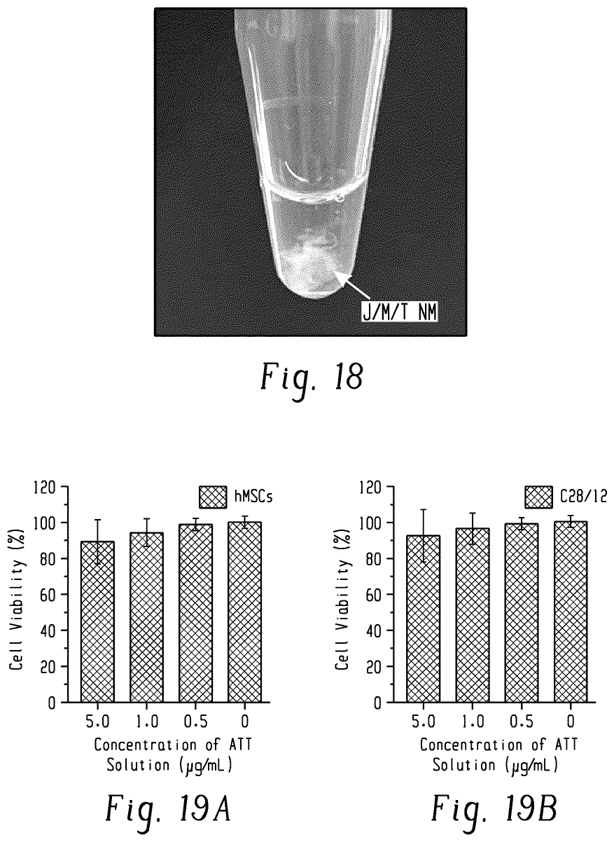

The cytotoxicity of the JBNTs was evaluated using the cell counting kit-8 (CCK-8) assay. hMSCs and human chondrocytes was cultured with JBNTs for 24 hours. The concentration of the JBNT solution was set as a gradient at 5 μg mL⁻¹1, 1 μg mL⁻¹, 0.5 μg mL⁻¹, and 0 μg mL⁻¹. Even at the highest concentration, the JBNTs presented excellent cell viability (>88%) (FIG. 19). The excellent biocompatibility of the JBNTs may arise from their DNA-mimicking chemistry and non-covalent structure. Therefore, the JBNTs are safe for use in cartilage tissue construction.

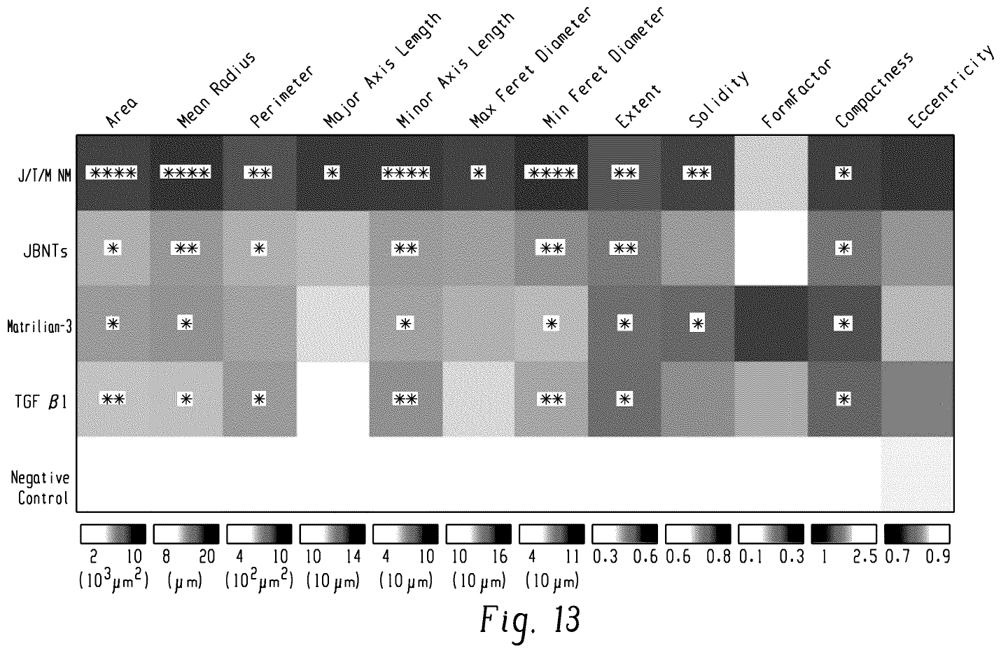

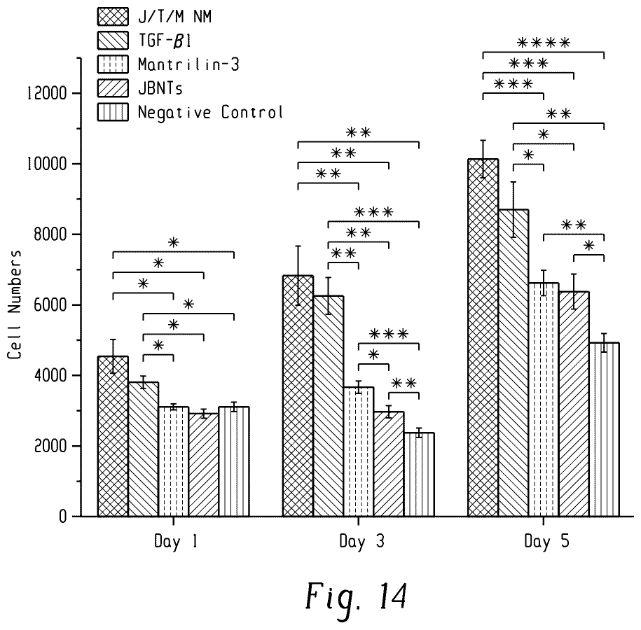

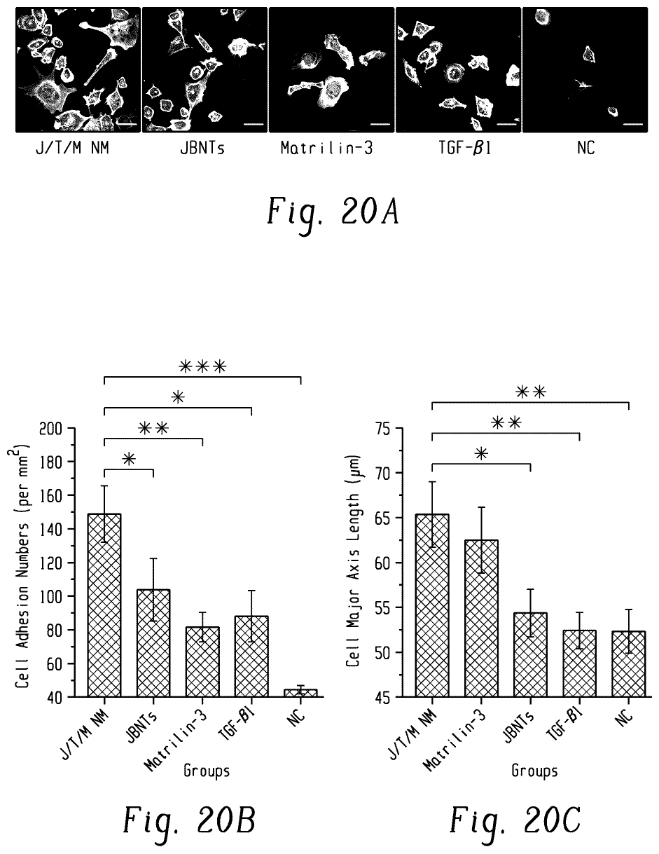

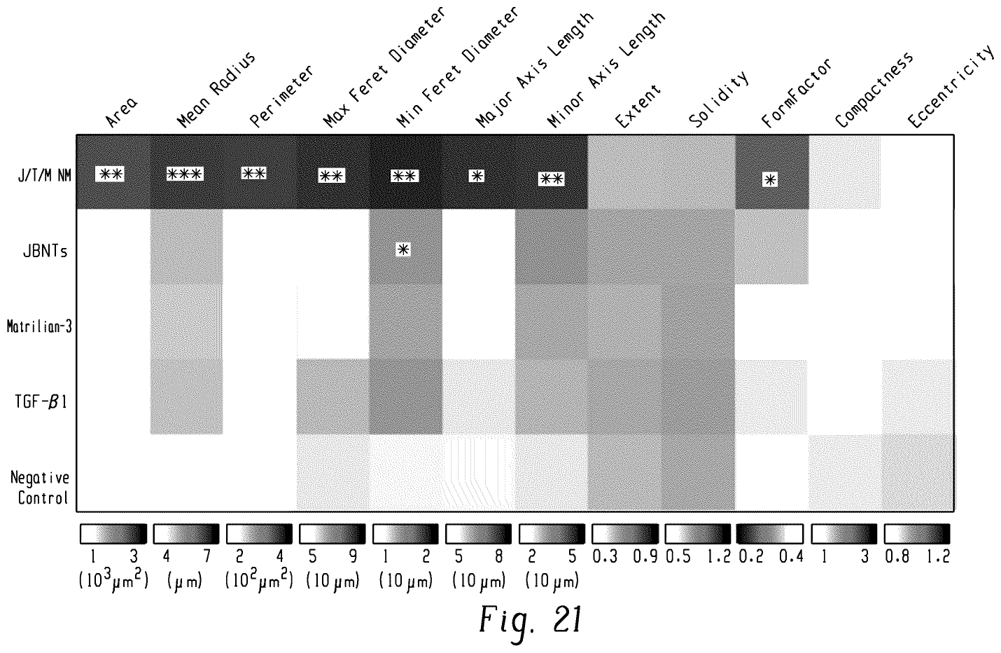

To determine the cell anchorage and adhesion ability of the J/T/M NM, the NMs were coated on the surface of agarose gel (a biocompatible but not bioactive material) and chambered coverglasses. We assessed the cell adhesion on these two surfaces. For the agarose surface, light microscopy indicated the extent of hMSC seeding on the agarose surface. As shown in FIG. 12A, many hMSCs clustered along the J/T/M NM. In the JBNTs group, there were a few cells anchored on the JBNT fibers as well. However, for the other groups, the hMSCs were evenly distributed without obvious alignment. The different behavior of the hMSCs provides direct evidence that the J/T/M NM dramatically enhances cell anchorage on its scaffold fibers. For the chambered coverglass surface, confocal images of hMSCs and human chondrocytes indicated the level of cell adhesion and morphology. The cells cultured with the J/T/M NM appeared to be more stretched than the other groups, indicating that these cells have excellent affinity with the J/T/M NM surface (FIG. 12B, FIG. 20A). The number of adhered cells and the cell major axis length in the confocal images were also analyzed. As shown in FIG. 12C and FIG. 20B, after cultivation with different biomaterials for 4 hours, the J/T/M NM group showed significantly higher cell adhesion density than the other groups. The average major axis length of cells in the J/T/M NM group was significantly larger than those in the JBNT, TGF-β1, and negative control groups (FIG. 12D, FIG. 20C). Furthermore, a thorough cell morphological analysis was conducted to elucidate the differences among the various materials. Twelve cell shape parameters via Cell Profiler were quantified, and statistical analyses were performed to evaluate the effect of each material. The obtained statistical analysis map indicates that the J/T/M NM surface group had the highest bioactivity and generated the most significant effect among all of the groups for both hMSCs and human chondrocytes (FIG. 13, FIG. 21). The bioactivity of the J/T/M NM is a synergistic effect of the JBNTs, TGF-β1, and matrilin-3, but does not result from a simple addition of their effects.

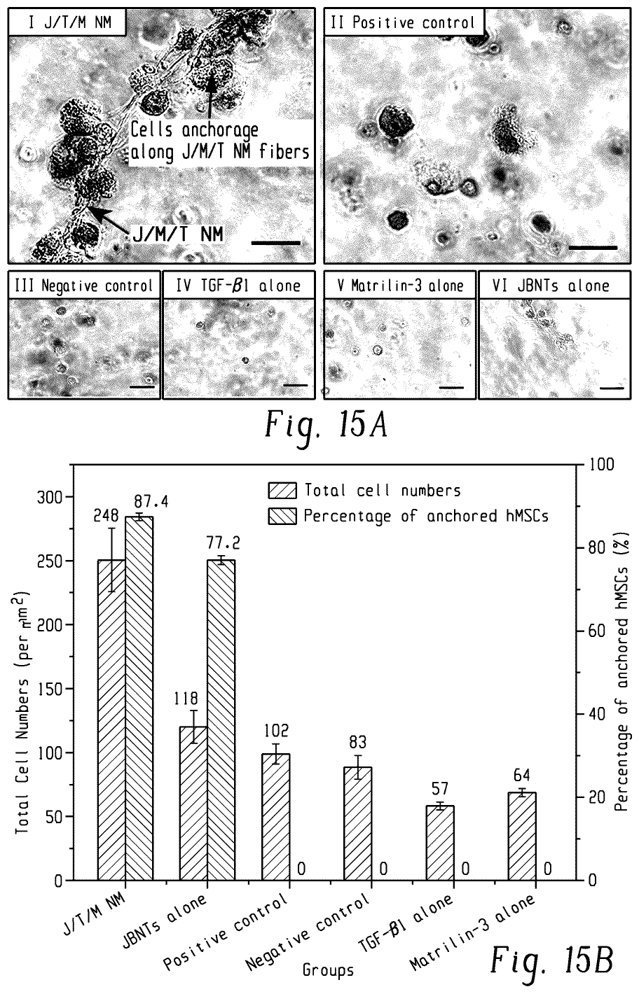

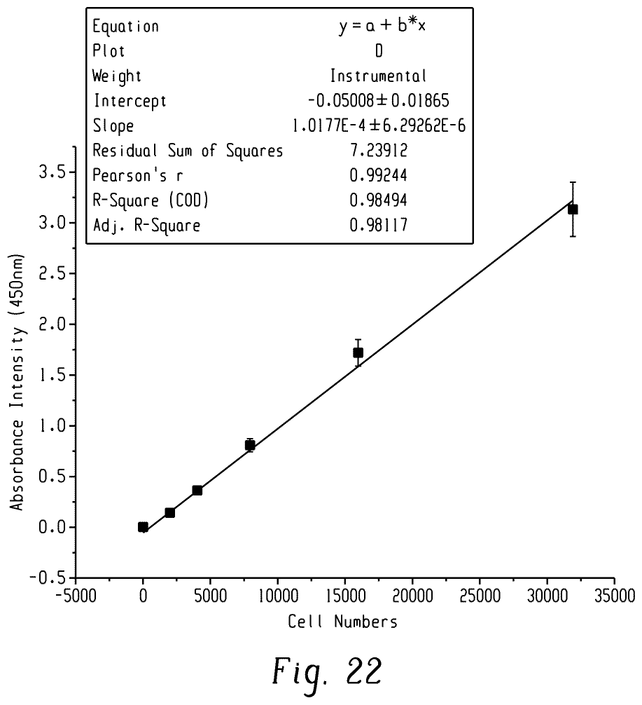

The ability of the NM to increase cell proliferation also was explored. After one day of cell culture with different materials, the J/T/M NM and TGF-β1 groups showed significantly higher cell numbers than the matrilin-3, JBNT, and negative control groups. When the cell culture time was increased to 3 or 5 days, the J/T/M NM and TGF-β1 groups demonstrated more obvious effects related to the promotion of cell proliferation than the other three groups. The bioactivity promoting cell proliferation was primarily attributed to the contribution of TGF-β1, as TGF-β1 is a growth factor, which can increase cell proliferation and differentiation. Additionally, matrilin-3 is a cartilage-specific protein, and JBNTs mimic the ECM. Both of these proteins can moderately increase cell proliferation after incubation with hMSCs for 3 or 5 days (FIG. 14, FIG. 22).

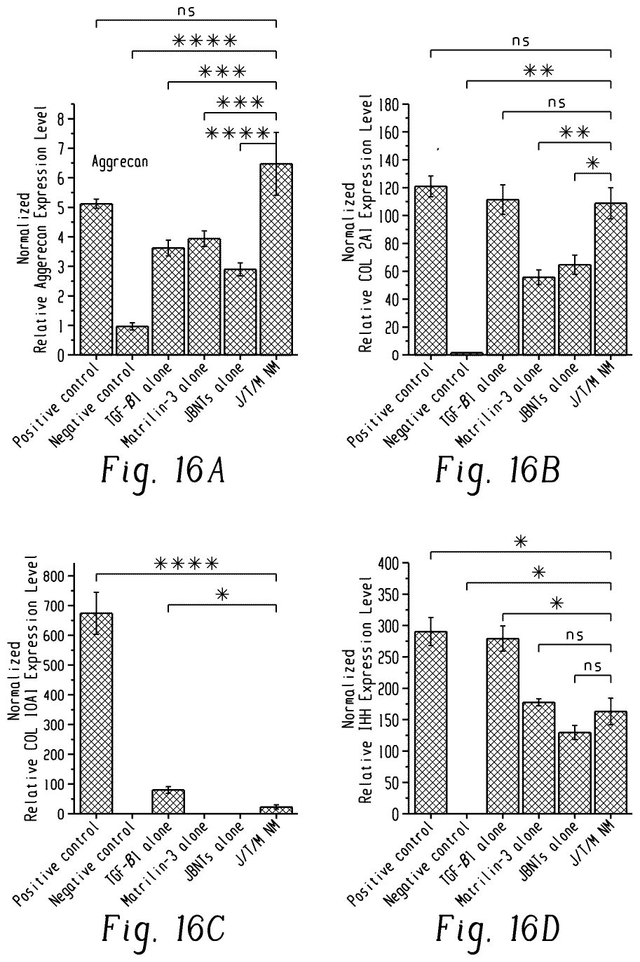

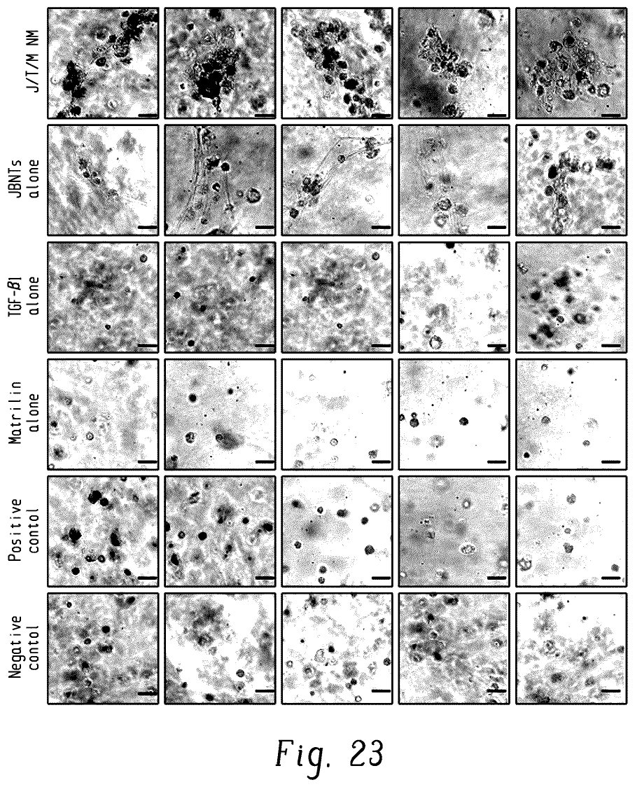

As a long-term function study, stem cells were cultured with the J/T/M NM or other materials in three-dimensional cartilage tissue constructs. The positive control group was supplied with fresh TGF-β1 each time the medium was changed. The medium was changed every three days. The same doses of TGF-β1, matrilin-3, and JBNTs applied in the J/T/M NM were encapsulated in agarose to form tissue constructs, denoted as the TGF-β1, matrilin-3, JBNT, and J/T/M NM groups, respectively. After 15 days, the protein markers in the chondrogenic differentiation of hMSCs were evaluated. Alcian blue staining was applied to identify cartilage-specific proteoglycan expression in the cartilage tissue constructs. FIG. 15A(I) showed that the cells in the J/T/M NM group were stained with blue, providing additional evidence for the enhanced chondrogenesis of hMSCs after culturing with the J/T/M NM. Importantly, hMSCs clustered alongside the J/T/M NM bundles and proceeded through chondrogenic differentiation. In accordance with the adhesion study and our material design, the layer-by-layer NM not only provided ideal anchor sites for stem cells but also achieved localized bioactivity of TGF-β1 to induce chondrogenic differentiation along the J/T/M NM. In contrast, the positive control group showed some chondrogenesis but no cell alignment (FIG. 15A(II)). Important to note TGF-β1 needs to bind with cell surface receptors to be bioactive. When cells adhered and grew along NMs, they can digest/degrade JBNTs easily. Due to the DNA mimicking structure, JBNTs can disassemble into small-molecule units triggered by low pH or enzyme (such as uptaken by cells). Therefore, when cells grew along NMs, they can gradually degrade JBNTs and expose the enveloped TGF-β1. This may be another reason why hMSCs preferred to grow long NMs (FIG. 7a). Interestingly, the nature ECMs have a similar mechanism to preserve TGF-β1. Moreover, the other groups presented no or low chondrogenic differentiation, as demonstrated by the negative or very weak staining of Alcian blue (FIGS. 15A(III)-(VI)). Interestingly, although the same dose of TGF-β1 was added to both the TGF-β1-only group and the J/T/M NM group, only the J/T/M NM group showed a significant effect on the promotion of chondrogenesis (FIGS. 7A(I), 7A(IV)). This result confirms that the TGF-β1 encapsulated in the layer-by-layer structure presents a higher long-term bioactivity. Another interesting finding for the JBNTs-only group was observed: although JBNTs alone did not induce chondrogenesis, hMSCs preferred to adhere on the JBNT fibers, confirming that the JBNTs significantly promote stem cell anchorage (FIG. 15A(VI)). This finding is important for cartilage tissue engineering because a successful tissue scaffold should be able retain stem cells or cartilage cells at the desired location (such as a cartilage defect) for regeneration. The percentage of hMSCs anchored on JBNT fibers and the cell density was analyzed based on the light microscopy images. In J/T/M NM group, 87.4% hMSCs cells clustered alongside the J/T/M NM bundles, which is higher than the JBNTs alone group (77.2%). The cell density of the J/T/M NM group was enhanced obviously than other groups, which is another strong evidence that J/T/M NM has excellent ability on promoting cell adhesion and proliferation (FIG. 15B, FIG. 23).

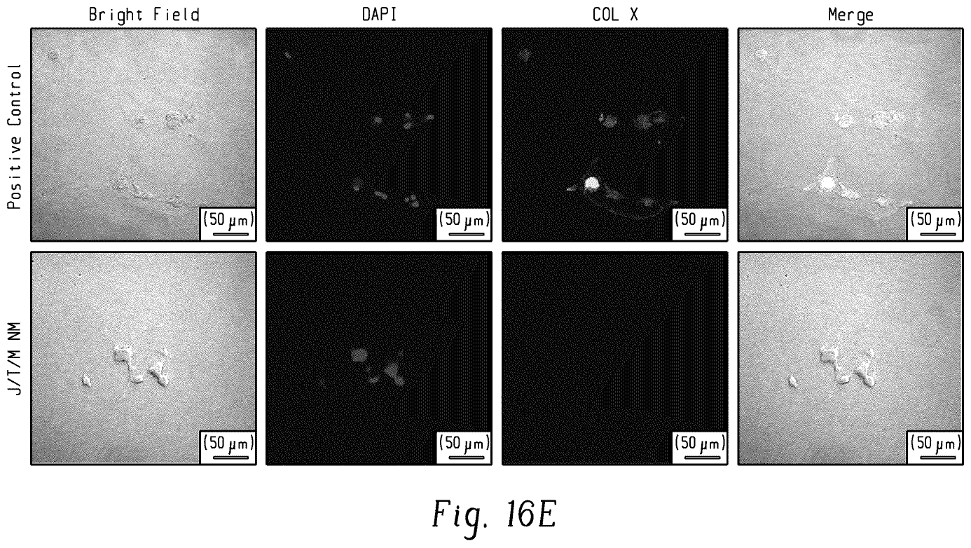

We investigated the chondrogenic differentiation markers and the hypertrophy markers. As shown in FIGS. 16A-16B, the J/T/M NM group has dramatically higher aggrecan and type II collagen (COL2A1) gene expression than the negative controls, demonstrating that the J/T/M NM can significantly promote stem cell chondrogenic differentiation. Also, the Alcian blue staining (FIG. 15) demonstrated the protein level of aggrecan in the J/T/M/NM group is significantly higher than other groups, consistent with the gene expression results. Importantly, the J/T/M NM group resulted in similar chondrogenesis ability compared with the positive control which was consistently supplied with fresh chondrogenic medium and TGF-β1, perhaps because the layer-by-layer structure of the NM stabilized TGF-β1 and subsequently resulted in long-lasting bioactivity of TGF-β1. This finding is important for cartilage tissue engineering, as free TGF-β1 has a short plasma half-life (<100 min). Moreover, although the positive control group promoted chondrogenesis, it presented poor homeostasis, as evidenced by the increased gene expression levels of hypertrophy markers (type X collagen and Indian hedgehog (IHH)) in the differentiated cells (FIGS. 16C-16D). In addition, the immunostaining of the cartilage constructs confirmed that the positive control group expressed a significant amount of type X collagen while the J/T/M NM group had minimal type X collagen staining (FIG. 16E). In contrast, the J/T/M NM tissue construct (with the anti-hypertrophy property provided by the JBNT/matrilin-3 microenvironment) exhibited a strong ability to prevent hypertrophy, which is another important property for cartilage tissue construct because a successful cartilage tissue construct should maintain homeostasis in long-term.

Herein, an injectable layer-by-layer J/T/M NM for cartilage tissue constructs was developed. This innovative layer-by-layer structure was realized by controlled self-assembly so that this highly organized scaffold was formed from the molecular level (which is at a smaller scale than conventional 3D printing and electrospinning techniques). The confined TGF-β1 in the inner layer of the matrix fibers to prevent its leakage to undesired locations and to promote localized chondrogenesis. Moreover, matrilin-3 was localized in the outer layer of the matrix fibers to create an anti-hypertrophic microenvironment. The JBNTs not only served as scaffold structural backbones but also enhanced stem cell anchorage and adhesion to localize cells along the scaffold fibers. In this manner, both stem cells and the growth factor (TGF-β1) were localized along the NM fibers. Therefore, the NM realized a homeostatic microenvironment for cartilage tissue regeneration at a confined location. In addition, the layer-by-layer J/T/M NM is injectable. Although the NM is a solid scaffold, it presented excellent structural flexibility (due to the DNA-mimicking JBNT backbones) in an aqueous environment and can be injected through a pipette tip, which can be broadly used for different scenarios (such as difficult-to-reach locations and irregularly shaped fractures or cavities). In the future, because JBNTs were demonstrated to be able to incorporate many different types of proteins or therapeutics, the layer-by-layer design of JBNT-based NMs can be customized for applications in various tissues. In sum, a NM was developed that is a promising platform for advanced cartilage tissue constructs.

### Experimental Section

Materials. JBNTs were synthesized by an approach that was previously published and shown to be effective. The hMSC Stem Cell Growth Medium Bullet Kit was obtained from Lonza. The C28/I2 human chondrocyte cell line was purchased from Millipore Sigma.

The hMSC Chondrogenic Differentiation Medium BulletKit™ was purchased from Lonza (catalog number PT-3003). Recombinant human matrilin-3 protein was purchased from R&D Systems. Recombinant human TGF-β1 (CHO derived) was purchased from PeproTech. DMEM, high-glucose cell culture medium (Gibco), trypsin-EDTA solution (0.25%, Gibco), ethanol (70% solution), 1× phosphate buffered saline (PBS, Gibco), fetal bovine serum (FBS, Gibco), penicillin-streptomycin (Gibco, 10,000 U mL⁻¹), and the Alcian blue stain kit (pH 2.5, Vector) were purchased from Fisher Scientific. Triton X-100 (Invitrogen, 1.0%), fixative solution (4% formaldehyde prepared in PBS), distilled water, DAPI nucleic acid stain (Invitrogen), rhodamine phalloidin (Invitrogen), Alexa Fluor® 488 Microscale Protein Labeling Kit, Alexa Fluor 555 Microscale Protein Labeling Kit, Human TGF beta 1 Elisa Kit (catalog number BMS249-4), Collagen X Antibody (catalog number PAS-97603), Goat anti-Rabbit IgG (H+L) Highly Cross-Adsorbed Secondary Antibody, Alexa Fluor® Plus 488 (Catalog number A32731), 10% Normal Goat Serum (Catalog number 50062Z), Fluoromount-G™ Mounting Medium (Catalog number 00-4958-02), and TRIzol™ reagent were purchased from Thermo Fisher Scientific. The CCK-8 assay was purchased from Millipore Sigma. Agarose (gel point 36° C.) was purchased from Sigma-Aldrich (catalog number A9539). The RNeasy® Plant Mini Kit was purchased from QIAGEN. The iTaq™ Universal SYBR Green One-Step Kit was purchased from Bio-Rad.

The 24-well and 96-well flat bottom cell culture plates (Corning) were obtained from Fisher Scientific (catalog numbers 07-200-740 and 07-200-91, respectively). Non-treated 96-well plates were purchased form Thermo Fischer Scientific (catalog number 260887), and 384-well assay plates were purchased from Corning (product number 3575). Number 1.5 glass coverslips were purchased from Thermo Fisher Scientific (catalog number 152250). The 8-well chambered coverglass systems were purchased from Thermo Fisher Scientific (catalog number 155409PK). Disposable cuvettes were purchased from Fisher Scientific (catalog number 14-955-128).

Multi-mode microplate readers (SpectraMax®, M3) were used to measure absorption values and the fluorescence spectra. A Nanodrop spectrophotometer (NanoDrop™ One/One^{c} UV-Vis) was used to measure the absorption spectra. Lab6 20-120 kV TEM was applied to obtain high-resolution images of the samples. A Leica SP8 spectral confocal microscope was used to obtain images for fluorescent dye-labeled NM. A Nikon A1R spectral confocal microscope was used to obtain fluorescent images of cells. A Zetasizer Nano ZS (Malvern Panalytical) was used to measure the zeta-potential of samples. A PCR instrument (Bio-Rad) was used to perform real-time PCR.

Zeta-potential test. Three groups of samples were prepared. For the matrilin-3 group, 160 μL of 10 μg mL⁻¹ matrilin-3 was dispersed in 640 μL H₂O to obtain an 800 μL test solution. For the mixed matrilin-3/TGF-β1 group, 160 μL of 10 μg mL⁻¹ matrilin-3 was mixed with 40 μL of 10 μg mL⁻¹ TGF-β1 and pipetted several times. The mixture was then dispersed in 600 μL H₂O to obtain an 800 μL test solution. For the J/T/M NM group, 160 μL of 10 μg mL⁻¹ matrilin-3 was mixed with 40 μL of 10 μg mL⁻¹ TGF-β1 and pipetted several times. Then, 20 μL of 1 mg mL⁻¹ JBNTs was added to the solution and pipetted several times. Finally, the J/T/M NM solution was dispersed in 580 μL H₂O to obtain an 800 μL test solution. The zeta-potential values of the three groups of samples were tested with a Zetasizer Nano ZS.

UV-Vis absorption spectra measurement. Four groups of samples were prepared. For the JBNT group, 5 μL of 1 mg mL⁻¹ JBNTs was added to 50 μL H₂O to obtain a 100 μg mL⁻¹ JBNT solution. For the matrilin-3 group, 40 μL of 10 μg mL⁻¹ matrilin-3 was added to 15 μL H₂O to obtain a 7.3 μg mL⁻¹ matrilin-3 solution. For the TGF-β1 group, 10 μL of 10 μg mL⁻¹ TGF-β1 was added to 45 μL H₂O to obtain a 1.8 μg mL⁻¹ TGF-β1 solution. For the J/T/M NM group, 40 μL of 10 μg mL⁻¹ matrilin-3 was mixed with 10 μL of 10 μg mL⁻¹ TGF-β1. Then, 5 μL of 1 mg mL⁻¹ JBNTs was added to the mixture solution and pipetted several times. The final concentrations of the JBNT, matrilin-3, and TGF-β1 samples were 100 μg mL⁻¹, 7.3 μg mL⁻¹, and 1.8 μg mL⁻¹, respectively. The absorption spectrum of each group of samples was measured with a NanoDrop spectrophotometer.

TEM characterization. First, 10 μL of 1 mg mL⁻¹ JBNTs was diluted with 40 μL distilled water to obtain a 200 μg mL⁻¹ JBNT solution. Next, 30 μL of 100 μg mL⁻¹ matrilin-3 was mixed with 20 μL of 100 μg mL⁻¹ TGF-β1 and pipetted several times. Finally, 10 μL of 1 mg mL⁻¹ JBNTs was added to the mixture solution to prepare the J/T/M NM sample. Two pieces of grids were cleaned with a Plasma Cleaner Harrick Plasma PDC-32G before negative staining was performed. The negative staining process was carried out for the specimens as follows: 3 μL JBNT solution (200 μg mL⁻¹) and 3 μL J/T/M NM solution were each dropped on separate grids and left for 2 min. Then, 100 μL uranyl acetate solution (0.5%) was pipetted onto the solution to rinse each grid. Excess solution was removed from the grids with filter paper and the grids were air-dried. Finally, Lab6 20-120 kV TEM was carried out for specimen characterization.

Video recordings of J/T/M NM fabrication. First, 8 μL TGF-β1 in a 1 mg mL⁻¹ aqueous solution was added to 32 μL of 1 mg mL⁻¹ matrilin-3 aqueous solution and pipetted several times. Then, 80 μL of JBNTs in a 1 mg mL⁻¹ aqueous solution was added to the TGF-β1/matrilin-3 mixture solution and pipetted several times. White floccules were produced immediately after the addition of JBNTs. 2-200 μL pipette tips (with a same tip diameter as 19 gauge needles) were used for NM injection. Video recordings captured the process of self-assembly.

Fluorescent spectra measurement. TGF-β1 was labeled using the Alexa Fluor® 488 Microscale Protein Labeling Kit. The final concentration of fluorescent dye-labeled TGF-β1 (TGF-β1-Alexa Fluor® 488) was 20 μg mL⁻¹. Matrilin-3 was labeled using the Alexa Fluor 555 Microscale Protein Labeling Kit. The final concentration of fluorescent dye-labeled matrilin-3 (matrilin-3-Alexa Fluor® 555) was 80 μg mL⁻¹.

Five groups of samples were prepared. For the JBNT group, 5 μL of 1 mg mL⁻¹ JBNTs was added to 40 μL H₂O to obtain a 111 μg mL⁻¹ JBNT solution. For the TGF-β1-Alexa Fluor® 488 group, 20 μL of 20 μg mL⁻¹ TGF-β1-Alexa Fluor® 488 was dispersed in 25 μL H₂O to obtain an 8.9 μg mL⁻¹ test solution. For the matrilin-3-555 group, 20 μL of 80 μg mL⁻¹ matrilin-3 was dispersed in 25 μL H₂O to obtain a 36 μg mL⁻¹ test solution. For the TGF-β-Alexa Fluor® 488/matrilin-3-Alexa Fluor® 555 mixed group, 20 μL of 80 μg mL⁻¹ matrilin-3-Alexa Fluor® 555 was mixed with 20 μL of 20 μg mL⁻¹ TGF-β1-Alexa Fluor® 488, and 5 μL H₂O. Then, the mixture solution was pipetted several times. For the TGF-β-Alexa Fluor® 488/matrilin-3-Alexa Fluor 555/JBNT NM group, 20 μL of 20 μg mL⁻¹ TGF-β1-Alexa Fluor® 488 was mixed with 20 μL of 80 μg mL⁻¹ matrilin-3-Alexa Fluor® 555 and pipetted several times. Then, 5 μL of 1 mg mL⁻¹ JBNTs was added to the solution and pipetted several times. The final concentrations of the JBNT, TGF-β1-Alexa Fluor® 488, and matrilin-β-Alexa Fluor® 555 samples were 111 μg mL⁻¹, 8.9 μg mL⁻¹, and 36 μg mL⁻¹, respectively.

Each sample group was added to one well of a black 384-well plate. The fluorescence spectra of the samples were measured with multi-mode microplate readers. The excitation wavelength for the measurements was 488 nm.

Confocal imaging for fluorescent dye-labeled NM. For confocal imaging, 10 μL of 20 μg mL⁻¹ TGF-β1-Alexa Fluor ®488 was mixed with 10 μL of 80 μg mL⁻¹ matrilin-3-Alexa Fluor® 555 and pipetted several times. Then, 5 μL 1 mg mL⁻¹ of JBNTs was added to the mixture solution and pipetted several times to develop the TGF-β-Alexa Fluor® 488/matrilin-3-Alexa Fluor® 555/JBNT NM. Some red floccules could be seen in the solution, due to aggregated NM. Then, 10 μL of the NM solution containing the red floccules was placed on a number 1.5 glass coverslip. Excess solution was removed with filter paper, while the red floccules remained on the coverslip. Confocal images were taken for the NM with a Leica SP8 spectral confocal microscope.

J/T/M NM stability test. Human TGF beta 1 ELISA Kit was used to test the release percentage of TGF-β1 from J/T/M NM in agarose hydrogel. The J/T/M NM in agarose hydrogel was prepared as follows. 10 μl of 10 μg mL⁻¹ TGF-β1 was mixed with 40 μl of 10 μg mL⁻¹ matrilin-3, 5 μl of 1 mg mL⁻¹ JBNTs, and 195 μl PBS to make J/T/M NM solution. Then the J/T/M NM solution was mixed with 250 μL 2% agarose to get the J/T/M NM agarose hydrogel. 500 μl PBS was used as a release solution and changed every 3 days. After 15 days, all collected release solutions were tested with an ELISA kit. UV-Vis absorption spectra of J/T/M NM solution were tested every 3 days to explore its stability. JBNTs solution, matrilin-3 solution, TGF-β1 solution, and JBNTs+matrilin-3 solutions were used as control groups.

Cytotoxicity assay. hMSCs and human chondrocyte cells were seeded onto two separate 96-well plates. Each well of the plates received 100 μL of cell suspension containing 5,000 cells. The two plates were incubated in a cell culture incubator for 24 h (37° C., 5% CO₂). Each well then received 100 μL of various concentrations of JBNTs diluted in distilled water. The JBNT concentration gradient was set as 5 μg mL⁻¹, 1 μg mL⁻¹, 0.5 μg mL⁻¹, and 0 μg mL⁻¹. For each group, 6 wells were used for testing. After a 24 h incubation, each well received 10 μL of CCK-8 solution and were incubated for an additional 2 h. The absorption values of the plates were measured with multi-mode microplate readers at 450 nm.

Adhesion test on pre-coated agarose. Five groups of samples were prepared. For the JBNT group, 10 μL of 1 mg mL⁻¹ JBNTs was added to 230 μL distilled water to obtain a 240 μL JBNT solution. For the TGF-β1 group, 20 μL of 100 μg mL⁻¹ TGF-β1 was dispersed in 220 μL distilled water. For the matrilin-3 group, 80 μL of 100 μg mL⁻¹ matrilin-3 was dispersed in 160 μL distilled water. For the J/T/M NM group, 20 μL of 100 μg mL⁻¹ TGF-β1 was mixed with 80 μL of 100 μg mL⁻¹ matrilin-3 and pipetted several times. Then, 10 μL of 1 mg mL⁻¹ JBNTs was added to the solution. After the solution was pipetted several times, it was diluted with 130 μL distilled water. As the control group, 240 μL distilled water was used. To coat the 96-well plates, a 1.0 wt % agarose solution was prepared. After the plates were coated with agarose for 10 min, 6 wells were designated for each group. Each well received 40 μl sample and 70 μL agarose. The plates were placed in a biosafety cabinet and air-dried for 5 h. Then, 100 μL hMSC suspensions containing 8,000 cells were added to each well with samples. The cells were incubated for 40 min (37° C., 5% CO₂). The cell culture medium was replaced to remove unattached cells. Pictures of the attached cells were taken for each group using a light microscope.

Adhesion test on pre-coated coverglass chambers. Two chambered coverglasses were prepared for the adhesion experiments. Each chambered coverglass included five groups of samples. For the JBNT group, 1.25 μL of 1 mg mL⁻¹ JBNTs was diluted with 198.75 μL distilled water to obtain a 200 μL solution. The concentration of the JBNT solution was 6.25 μg mL⁻¹. For the matrilin-3 group, 10 μL of 10 μg mL⁻¹ matrilin-3 was diluted with 190 μL distilled water to obtain a 0.5 μg mL⁻¹ solution. For the TGF-β1 group, 2.5 μL of 10 μg mL⁻¹ TGF-β1 was diluted with 197.5 μg mL⁻¹ distilled water to obtain a 0.125 μg mL⁻¹ solution. For the J/T/M NM group, 10 μL of 10 μg mL⁻¹ matrilin-3 was mixed with 2.5 μL of 10 μg mL⁻¹ TGF-β1 and pipetted several times. Next, 1.25 μL of 1 mg mL⁻¹ JBNTs was added to the mixture solution and pipetted. Then, 186.25 μL distilled water was added to obtain a 200 μL NM solution. As a control group, 200 μL distilled water was used. Each sample group was added into one well of a Number 1.5 chambered coverglass. The chambered coverglasses were placed into a −80° C. freezer for one hour and then freeze-dried with a lyophilized instrument.

hMSCs and human chondrocyte cells (10,000 cells/each well) were seeded onto separated chambered coverglasses. The two chambered coverglasses were incubated in a 37° C. incubator for 4 h. Then, the cell culture medium was pipetted out and the cells were rinsed twice with PBS. The cells were fixed with 4% paraformaldehyde for 5 min. The fixative solution was removed, the cells were rinsed twice with PBS, and the cells were incubated with 100 μL 0.1% Triton™-X for 10 min. After two rinses with PBS, 100 μL of 0.165 μM rhodamine-phalloidin was added to each well for 30 min. Next, 0.1 μg mL⁻¹ DAPI was used to stain the cell nuclei. After a 5 min incubation, the DAPI was pipetted out. Finally, the cells were rinsed twice with PBS.

A Nikon A1R spectral confocal microscope was used to observe the morphology and obtain fluorescent images of cells. Analyses of the number and morphology of cells were performed with Cell Profiler, MATLAB, and Image J.

Cell proliferation. Five groups of samples were added to an untreated 96-well plate for the cell proliferation experiment. For the JBNT group, 3.75 μL of 1 mg mL⁻¹ JBNTs was added to 596.25 μL distilled water to obtain a 600 μL JBNT solution. The concentration of the solution was 6.25 μg mL⁻¹. For the TGF-β1 group, 7.5 μL of 10 μg mL⁻¹ TGF-β1 was dispersed in 592.5 μL distilled water to obtain a 0.125 μg mL⁻¹ test solution. For the matrilin-3 group, 30 μL of 10 μg mL⁻¹ matrilin-3 was dispersed in 570 μL distilled water to obtain a 0.5 μg mL⁻¹ test solution. For the J/T/M NM group, 7.5 μL of 10 μg mL⁻¹ TGF-β1 was mixed with 30 μL of 10 μg mL⁻¹ matrilin-3 and pipetted several times. Then, 3.75 μL of 1 mg mL⁻¹ JBNTs was added to the solution and pipetted. The J/T/M NM solution was diluted with 558.75 μL distilled water. The final concentrations of JBNT, TGF-β1, and matrilin-3 samples were 6.25 μg mL⁻¹, 0.125 μg mL⁻¹, and 0.5 μg mL⁻¹, respectively. As a control group, 600 μL distilled water was used. Each sample group was divided into 6 wells, with each well receiving a 100 μL sample. The three plates were placed into a −80° C. freezer for one hour and then freeze-dried with a lyophilized instrument.

hMSCs were seeded onto these plates. Each well received 100 μL cell suspension containing 5,000 cells. Three plates were incubated at 37° C. for 1 day, 3 days, or 5 days (5% CO₂). After incubation, 10 μL CCK-8 solution was added to each well with cells. Then, each plate was incubated at 37° C. for another 2 h. The absorption values of the plates were measured with multi-mode microplate readers at 450 nm. A series of a known number of hMSCs were seeded onto a 96-well plate. After incubating for 4 h, the absorption values of the cells were measured with the CCK-8 assay. A standard curve was generated according to the absorption values and the numbers of hMSCs. Cell proliferation was calculated with the absorption standard curve.

Cell differentiation test with Real-time PCR. Seven groups of samples were prepared. For each group, 20 μL cell suspension (4×10⁴ cells) was mixed with 30 μL solution/PBS and 50 μL of 2.0 wt % agarose to make one agarose tissue construct. The solutions were prepared as follows. For the TGF-β1 group, 5 μL of 10 μg mL⁻¹ TGF-β1 was diluted with 25 μL PBS to obtain a 1.6 μg mL⁻¹ solution. For the matrilin-3 group, 20 μL of 10 μg mL⁻¹ matrilin-3 was diluted with 10 μL PBS to obtain a 6.7 μg mL⁻¹ solution. For the JBNT group, 2.5 μL of 1 mg mL⁻¹ JBNTs was diluted with 27.5 μL PBS to obtain an 83.3 μg mL⁻¹ solution. For the J/T/M NM group, 20 μL of 10 μg mL⁻¹ matrilin-3 was mixed with 5 μL of 10 μg mL⁻¹ TGF-β1 and the mixture was pipetted several times. Then, 2.5 μL of 1 mg mL⁻¹ JBNTs was added to the mixture solution and pipetted, followed by a dilution with 2.5 μL PBS. Finally, 30 μL PBS was used to replace the samples for each control group.

For each well, 0.5 mL cell culture medium was added and the medium was replaced every three days. For the positive control group, commercial hMSC chondrogenic medium with additional TGF-β1 was used for cell culture. The same dose of TGF-β1 was used for the positive control and the TGF-β1 and the J/T/M NM group. For one negative control group, commercial hMSC chondrogenic differentiation cell culture medium without TGF-β1 was applied. For a second negative control group, DMEM cell culture medium with 10% FBS was applied. For other test groups, commercial hMSC chondrogenic cell culture medium without TGF-β1 was used. After 15 days, the tissue constructs were harvested. Total RNA was extracted with TRIzol™ reagent and the RNeasy® Plant Mini Kit. qPCR was carried out to analyze the differentiation.

Type X collagen expression assay with immunostaining. Agarose tissue constructs were prepared and cultured in the same way as what we did in the “Cell differentiation test with Real-time PCR” section. After 15 days, the tissue constructs were harvested and to be used for immunostaining and Alcian Blue staining. The tissue constructs were fixed with 4% formaldehyde for one day and then soaked in a 30% sucrose solution overnight. The optimal cutting temperature compound reagent was used to embed those tissue constructs. 20 μm frozen sections were prepared for immunostaining. The frozen sections were stained with Col X antibody and Alexa Fluor 488 labeled secondary antibody and then observed with confocal microscopy.

Cell differentiation test with Alcian Blue staining. Cell culture medium was pipetted out of each well. Each tissue construct was rinsed with PBS three times, and the tissue constructs were then fixed with 4% formaldehyde for 30 min. The fixative solution was removed and the tissue constructs were rinsed with PBS three times. Acetic acid solution was applied to the tissue constructs for 15 min. After excess solution was removed, Alcian blue solution was applied for 45 min at 37° C. The tissue construct was then rinsed with acetic acid solution three times to remove most of the excess Alcian Blue solution. The acetic acid solution was applied to the tissue constructs for one day at 37° C. and the solution was pipetted out several times to remove the remaining free dye. The hMSCs were imaged using microscopy.

Statistics. Data were expressed as the mean +/−standard error of the mean. Statistics were performed using student t-test for parametric data and ANOVA followed by Dunn's post-analysis for non-parametric data with p<0.05 considered statistically significant.

1. A self-assembled nanomaterial, comprising a Janus base nanotube having a biologically active molecule noncovalently adhered thereto, wherein the biologically active molecule comprises an extracellular matrix (ECM) molecule, a bioactive molecule, or a combination thereof.

2. The nanomaterial of claim 1, wherein the ECM molecule comprises hydroxyapatite, fibronectin, Matn1, MAtn3, laminin, a collagen, elastin, vitronectin, fibrillin, perlecan, fibrinogen, osteonectin, tenascin, thrombospondin, an intercellular adhesion molecule, an integrin, a proteoglycan, a glycoprotein, or a combination thereof.

3. The nanomaterial of claim 1, wherein the ECM molecule comprises type I collagen or type II collagen.

4. The nanomaterial of claim 1, wherein the ECM molecule comprises ICAM1-5.

5. The nanomaterial of claim 1, wherein the ECM molecule comprises aggrecan or a glycosaminoglycan.

6. The nanomaterial of claim 1, wherein the ECM molecule comprises hyaluronic acid, chondroitin sulfate, dermatin sulfate, keratan sulfate, heparin, or heparin sulfate.

7. The nanomaterial of claim 1, wherein the bioactive molecule comprises TGFβ, VEGF, IGF, EGF, PDGF, a BMPs, an FGF, GDNF, HGF, PGF, NGF, TNF-α, SDF-1, dexamethasone, an siRNA, an miRNA, a growth factor, a small-molecule drug, or a combination thereof.

8. The nanomaterial of claim 1, wherein the nanomaterial comprises only one type of Janus base nanotube and only one biologically active molecule, and the biologically active molecule is not hydroxyapatite or Matn3.

9. The nanomaterial of claim 1, wherein
(a) the Janus base nanotube comprises a compound of Formula (I): 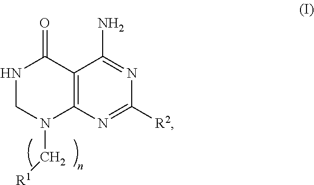
or a pharmaceutically acceptable salt thereof, wherein:
n is 1, 2, 3, 4, 5, or 6;
R¹ is selected from an α-amino acid, a β-amino acid, an α-polypeptide, and a β-polypeptide; and
R² is selected from H, CH₃, and NHR^{z}, wherein R^{z} is H or a C₁ to C₂₀ aliphatic group;
(b) the Janus base nanotube comprises a compound of Formula (III): 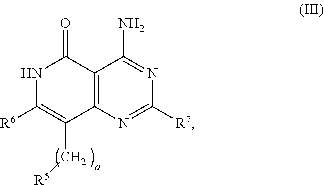
or a pharmaceutically acceptable salt thereof, wherein:
n is 1, 2, 3, 4, 5, or 6;
R⁵ is selected from an α-amino acid, a β-amino acid, an α-polypeptide and a β-polypeptide,
R⁶ and R⁷ are each independently selected from H, CH₃, and NHR^{z}; and
R^{z} is H or a C₁ to C₂₀ aliphatic group;
(c) the Janus base nanotube comprises a compound of Formula (V): 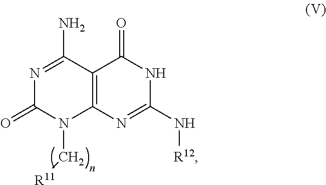
or a pharmaceutically acceptable salt or ester thereof, wherein:
n is 1, 2, 3, 4, 5, or 6;
R¹¹ is selected from an α-amino acid, a β-amino acid, an α-polypeptide and a β-polypeptide,
R², R⁶, and R⁷ are each independently selected from H, CH₃, and NHR^{z}; and
R^{z}, R¹², and R¹⁶ are each independently H or a C₁ to C₂₀ aliphatic group;
(d) the Janus base nanotube comprises a compound of Formula (VII): 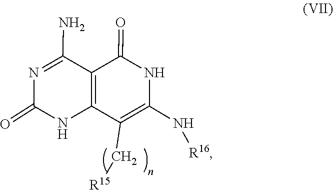
or a pharmaceutically acceptable salt or ester thereof, wherein:
n is 1, 2, 3, 4, 5, or 6;
R¹⁵ is selected from an α-amino acid, a β-amino acid, an α-polypeptide and a β-polypeptide, and
R¹⁶ is H or a C₁ to C₂₀ aliphatic group; or
(e) the Janus base nanotube comprises a compound of Formula (IX): 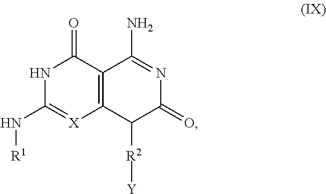
or a pharmaceutically acceptable salt, wherein:
X is CH or nitrogen;
R₂ is hydrogen or a C₁ to C₂₀ linker group;
Y is absent when R₂ is hydrogen, or is an amino acid or polypeptide having an amino group covalently bound to an α-carbon of the amino acid and the amino group is covalently bound to the linker group R₂; and
R¹ is hydrogen or C₁ to C₂₀ aliphatic moiety, such as alkyl, straight or branched chain, saturated or unsaturated alkyl.

10. (canceled)

11. (canceled)

12. (canceled)

13. The nanomaterial of claim 1, wherein
(a) the Janus base nanotube comprises a compound of Formula (II): 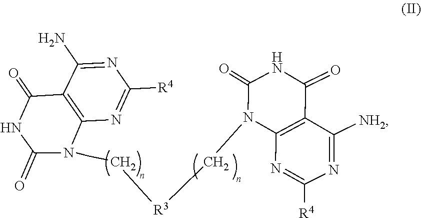
or a pharmaceutically acceptable salt thereof, wherein:
n is 1, 2, 3, 4, 5, or 6;
R³ is selected from an α-amino acid, a β-amino acid, an α-polypeptide, and a β-polypeptide;
R⁴ is H, CH₃, or NHR^{z}; and
R^{z} is H or a C₁ to C₂₀ aliphatic group;
(b) the Janus base nanotube comprises a compound of Formula (IV): 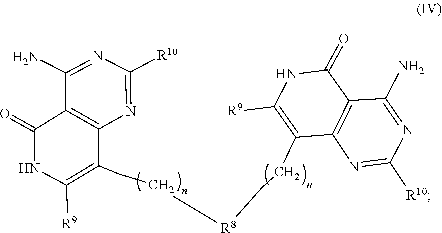
or a pharmaceutically acceptable salt thereof, wherein
n is 1, 2, 3, 4, 5, or 6;
R⁸ is selected from an α-amino acid, a β-amino acid, an α-polypeptide, and a β-polypeptide;
R⁹ and R¹⁰ are each independently H, CH₃, or NHR^{z}; and
R^{z} is H or a C₁ to C₂₀ aliphatic group;
(c) the Janus base nanotube comprises a compound of Formula (VI): 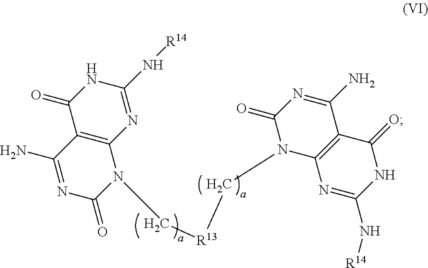
or a pharmaceutically acceptable salt thereof, wherein
n is 1, 2, 3, 4, 5, or 6;
R¹³ is selected from an α-amino acid, a β-amino acid, an α-polypeptide, and a β-polypeptide; and
R¹⁴ is H or a C₁ to C₂₀ aliphatic group; or
(d) the Janus base nanotube comprises a compound of Formula (VIII): 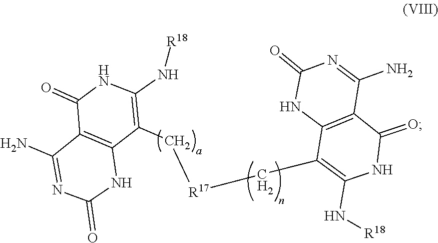
or a pharmaceutically acceptable salt thereof, wherein:
n is 1, 2, 3, 4, 5, or 6;
R¹⁷ is selected from an α-amino acid, a β-amino acid, an α-polypeptide, and a β-polypeptide; and
R¹⁸ is H or a C₁ to C₂₀ aliphatic group; or
(e) the Janus base nanotube comprises a compound of Formula (XI) 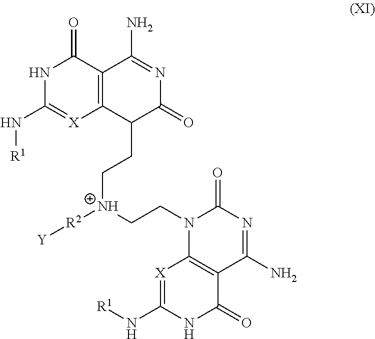
or a pharmaceutically acceptable salt, wherein:
X is CH or nitrogen;
R₂ is hydrogen or a C₁ to C₂₀ linker group;
Y is absent when R² is hydrogen, or is an amino acid or polypeptide having an amino group covalently bound to an α-carbon of the amino acid and the amino group is covalently bound to the linker group R²; and
R¹ is hydrogen or a C₁ to C₂₀ aliphatic moiety, such as alkyl, straight or branched chain, saturated or unsaturated.

14. (canceled)

15. (canceled)

16. (canceled)

17. (canceled)

18. (canceled)

19. The nanomaterial of claim 1, in the form of a single compartment nanomaterial.

20. The nanomaterial of claim 1, in the form of a multiple compartment nanomaterial.

21. The nanomaterial of claim 20, wherein the multiple compartment nanomaterial is a double compartment nanomaterial.

22. An injectable composition comprising the nanomaterial of claim 1, and a pharmaceutically acceptable carrier.

23. A tissue chip comprising a microfluidic cell and the nanomaterial of claim 1.

24. A method of tissue engineering, comprising injecting into a tissue the injectable composition of claim 22.

25. The method of claim 24, wherein the tissue is selected from cartilage, bone, brain, spine, joint, nerve, ligament and tendon, bone marrow, heart, eye, liver, kidney, and lung.
